(19) United States
(12) Patent Application Publication
(10) Pub. No.: US 2008/0269250 A1
(43) Pub. Date: Oct. 30, 2008

(54) Pyrrolidine and Piperidine Acetylene Derivatives for Use as Mglur5 Antagonists

(76) Inventors: Ralf Glatthar, Bad Saeckingen (DE); Thomas J. Troxler, Wahlen b. Laufen (CH); Thomas Zoller, Logelbach (FR); Joachim Nozulak, Heitersheim (DE)

Correspondence Address: NOVARTIS INSTITUTES FOR BIOMEDICAL RESEARCH, INC., 400 TECHNOLOGY SQUARE, CAMBRIDGE, MA, 02139, US

(21) Appl. No.: 11/816,853

(22) PCT Filed: Feb. 20, 2006
(86) PCT No.: PCT/EP2006/001505

§ 371 Date: Aug. 22, 2007

(30) Foreign Application Priority Data
Feb. 22, 2005 (GB) 0503646.2

(51) Int. Cl. A61K 31/497; C07D 405/02; A61K 31/4525; C07D 401/02; A61K 31/4545; C07D 401/04; A61K 31/444; C07D 403/02

(52) U.S. Cl. 514/255.05; 546/214; 514/326; 514/316; 546/191; 546/257; 514/334; 544/336

(57) ABSTRACT

The invention provides compounds of formula (I)

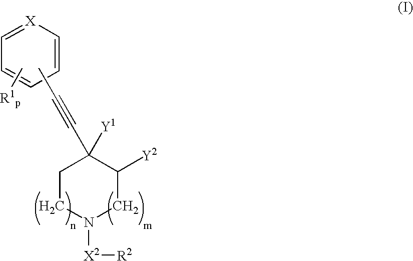

wherein the substituents are as defined in the description, processes and intermediates for their preparation and their use as pharmaceuticals.

The present invention relates to novel acetylene derivatives, their preparation, their use as pharmaceuticals and pharmaceutical compositions containing them.

More particularly the invention provides a compound of formula (I)

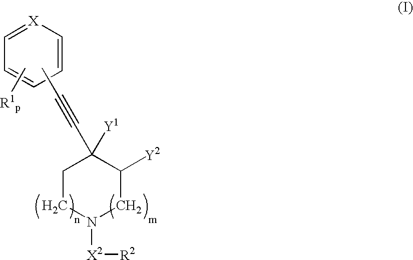

wherein
m represents 0 and n represents 1 or
m represents 0 and n represents 2 or
m represents 1 and n represents 1;
p represents 0, 1, 2, 3, 4 or 5;
X represents CH, N;
X² represents a single bond or an alkandiyl-group, optionally interrupted by one or more oxygen atoms or carbonyl groups or carbonyloxy groups
Y¹ represents OH and Y² represents H or
Y¹ and Y² form a bond;
R¹ represents halogen, cyano, nitro, —CHO, alkyl, alkoxy, halogenalkoxy, halogenalkyl, —C(O)R⁴, —COOR⁴ wherein R⁴ is alkyl or two substituents R¹ together form a alkandiyl or alkenediyl-moiety;
R² represents an unsubstituted or substituted heterocycle, or
R² represents phenyl or substituted phenyl, or
R² represents C(O)R³ wherein R³ represents alkyl, alkoxy or substituted alkoxy, phenyl or substituted phenyl, an unsubstituted or substituted aliphatic heterocycle, an unsubstituted or substituted partly saturated heterocycle containing less than 12 ring atoms, an unsubstituted or substituted aromatic heterocycle containing less than 12 ring atoms or
R² represents C(O)R³ wherein R³ represents unsubstituted or substituted cycloalkyl
R² represents CH₂R⁶, SR⁶, S(O)R⁶, S(O)₂R⁶ wherein R⁶ represents an unsubstituted or substituted heterocycle
   in free base or acid addition salt form.

In the present specification, the following definitions shall apply if no specific other definition is given:

“Alkyl” represents a straight-chain or branched-chain alkyl group, preferably represents a straight-chain or branched-chain C₁₋₁₂alkyl, particularly preferably represents a straight-chain or branched-chain C₁₋₆alkyl; for example, methyl, ethyl, n- or iso-propyl, n-, iso-, sec- or tert-butyl, n-pentyl, n-hexyl, n-heptyl, n-octyl, n-nonyl, n-decyl, n-undecyl, n-dodecyl, with particular preference given to methyl, ethyl, n-propyl and iso-propyl.

“Alkandiyl” represents a straight-chain or branched-chain alkandiyl group bound by two different Carbon atoms to the molecule, it preferably represents a straight-chain or branched-chain C₁₋₁₂ alkandiyl, particularly preferably represents a straight-chain or branched-chain C₁₋₆ alkandiyl; for example, methandiyl (—CH₂—), 1,2-ethanediyl (—CH₂—CH₂—), 1,1-ethanediyl ((—CH(CH₃)—), 1,1-, 1,2-, 1,3-propanediyl and 1,1-, 1,2-, 1,3-, 1,4-butanediyl, with particular preference given to methandiyl, 1,1-ethanediyl, 1,2-ethanediyl, 1,3-propanediyl, 1,4-butanediyl.

Each alkyl part of “alkoxy”, “alkoxyalkyl”, “alkoxycarbonyl”, “alkoxycarbonylalkyl” and “halogenalkyl” shall have the same meaning as described in the above-mentioned definition of “alkyl”.

“Alkenyl” represents a straight-chain or branched-chain alkenyl group, preferably C₂₋₆alkenyl, for example, vinyl, allyl, 1-propenyl, isopropenyl, 2-butenyl, 2-pentenyl, 2-hexenyl, etc. and preferably represents C₂₋₄ alkenyl.

“Alkendiyl” represents a straight-chain or branched-chain alkendiyl group bound by two different Carbon atoms to the molecule, it preferably represents a straight-chain or branched-chain C₂₋₆ alkandiyl; for example, —CH═CH—, —CH═C(CH₃)—, —CH═CH—CH₂—, —C(CH₃)═CH—CH₂—, —CH═C(CH₃)—CH₂—, —CH═CH—C(CH₃)H—, —CH═CH—CH═CH—, —C(CH₃)═CH—CH═CH—, —CH═C(CH₃)—CH═CH—, with particular preference given to —CH═CH—CH₂—, —CH═CH—CH═CH—.

“Alkynyl” represents a straight-chain or branched-chain alkynyl group, preferably C₂₋₆alkynyl, for example, ethenyl, propargyl, 1-propynyl, isopropenyl, 1-(2- or 3) butynyl, 1-(2- or 3) pentenyl, 1-(2- or 3) hexenyl, etc., preferably represents C₂₋₄alkynyl and particularly preferably represents ethynyl.

“Aryl” represents an aromatic hydrocarbon group, preferably a C₆-10 aromatic hydrocarbon group; for example phenyl, naphthyl, especially phenyl.

“Aralkyl” denotes an “Aryl” bound to an “Alkyl” (both as defined above) an represents, for example benzyl, α-methylbenzyl, 2-phenylethyl, α,α-dimethylbenzyl, especially benzyl.

“Heterocycle” represents a saturated, partly saturated or aromatic ring system containing at least one hetero atom. Preferably, heterocycles consist of 3 to 11 ring atoms of which 1-3 ring atoms are hetero atoms. Heterocycles may be present as a single ring system or as bicyclic or tricyclic ring systems; preferably as single ring system or as benz-annelated ring system. Bicyclic or tricyclic ring systems may be formed by annelation of two or more rings, by a bridging atom, e.g. Oxygen, sulfur, nitrogen or by a bridging group, e.g. alkandediyl or alkenediyl. A Heterocycle may be substituted by one or more substituents selected from the group consisting of Oxo (═O), Halogen, Nitro, Cyano, Alkyl, Alkandiyl, Alkenediyl, Alkoxy, Alkoxyalkyl, Alkoxycarbonyl, Alkoxycarbonylalkyl, Halogenalkyl, Aryl, Aryloxy, Arylalkyl. Examples of heterocyclic moieties are: pyrrole, pyrroline, pyrrolidine, pyrazole, pyrazoline, pyrazolidine, imidazole, imidazoline, imidazolidine, triazole, triazoline, triazolidine, tetrazole, furane, dihydrofurane, tetrahydrofurane, furazane (oxadiazole), dioxolane, thiophene, dihydrothiophene, tetrahydrothiophene, oxazole, oxazoline, oxazolidine, isoxazole, isoxazoline, isoxazolidine, thiazole, thiazoline, thiazolidine, isothiazole, isothiazoline, isothiazolidine, thiadiazole, thiadiazoline, thiadiazolidine, pyridine, piperidine, pyridazine, pyrazine, piperazine, triazine, pyrane, tetrahydropyrane, thiopyrane, tetrahydrothiopyrane, oxazine, thiazine, dioxine, morpholine, purine, pterine, and the corresponding benz-annelated heterocycles, e.g. indole, isoindole, cumarine, cumaronecinoline, isochinoline, cinnoline and the like.

“Hetero atoms” are atoms other than Carbon and Hydrogen, preferably Nitrogen (N), Oxygen (O) or Sulfur (S).

“Halogen” represents Fluoro, Chloro, Bromo or Iodo, preferably represents Fluoro, Chloro or Bromo and particularly preferably represents Chloro.

Compounds of formula (I) exist in free or acid addition salt form. In this specification, unless otherwise indicated, language such as “compounds of formula (I)” is to be understood as embracing the compounds in any form, for example free base or acid addition salt form. Salts which are unsuitable for pharmaceutical uses but which can be employed, for example, for the isolation or purification of free compounds of formula (I), such as picrates or perchlorates, are also included. For therapeutic use, only pharmaceutically acceptable salts or free compounds are employed (where applicable in the form of pharmaceutical preparations), and are therefore preferred.

On account of the asymmetrical carbon atom(s) that may be present in the compounds of formula (I) and their salts, the compounds may exist in optically active form or in form of mixtures of optical isomers, e.g. in form of racemic mixtures or diastereomeric mixtures. All optical isomers and their mixtures, including the racemic mixtures, are part of the present invention.

Preferred substituents, preferred ranges of numerical values or preferred ranges of the radicals present in the formula (I) and the corresponding intermediate compounds are defined below.
p preferably represents 0, 1 or 2.
p particularly preferably represents 1.
X preferably represents CH.
Y¹ preferably represents OH and Y² preferably represents H.
R¹ preferably represents halogen, cyano, nitro, —CHO, C₁₋₄ alkyl, C₁₋₄ alkoxy, halogen C₁₋₄ alkyl, —C(O)R⁴, —COOR⁴ wherein R⁴ is C₁₋₄ alkyl.
R¹ particularly preferably represents Fluoro, Chloro, Bromo, C₁₋₄ alkyl, C₁₋₄ alkoxy.
R¹ very particularly preferably represents Fluoro, Chloro, methyl, methoxy.

Further, two substituents R¹ preferably form one of the following groups:
—(CH₂)₄—, —(CH₂)₃—, —CH═CH—CH₂—, —CH═CH—CH═CH—.

Two substituents R¹ particularly preferably form one of the following groups:
—CH═CH—CH═CH—.
R² preferably represents an unsubstituted or substituted heterocycle having 3-11 ring atoms and 1-4 hetero atoms; the hetero atoms being selected from the group consisting of N, O, S, the substituents being selected from the group consisting of Oxo (═O), Halogen, Nitro, Cyano, C₁₋₄ Alkyl, C₁₋₄ Alkoxy, C₁₋₄ Alkoxyalkyl, C₁₋₄ Alkoxycarbonyl, C₁₋₄ Alkoxycarbonylalkyl, C₁₋₄ Halogenalkyl, C₆₋₁₀ Aryl, Halogen-C₆₋₁₀ Aryl, C₆₋₁₀ Aryloxy, C₆₋₁₀-Aryl-C₁₋₄ alkyl.
R² preferably represents phenyl or substituted phenyl, the substituents being selected from the group consisting of Halogen, Nitro, Cyano, C₁₋₄ Alkyl, C₁₋₄ Alkoxy, C₁₋₄ Alkoxyalkyl, C₁₋₄ Alkoxycarbonyl, C₁₋₄ Alkoxycarbonylalkyl, C₁₋₄ Halogenalkyl, C₆₋₁₀ Aryl, Halogen-C₆₋₁₀ Aryl, C₆₋₁₀ Aryloxy, C₆₋₁₀-Aryl-C₁₋₄ alkyl.
R² further preferably represents C(O)R³ wherein R³ represents C₁₋₄ alkyl; unsubstituted or substituted C₁₋₄ alkoxy, the substituents being selected from the group consisting of C₆₋₁₀ Aryl, Halogen-C₆₋₁₀ Aryl, C₁₋₄Alkyl —C₆₋₁₀ Aryl, C₁₋₄Alkoxy —C₆₋₁₀ Aryl, C₁₋₄Halogenalkyl —C₆₋₁₀ Aryl; unsubstituted or substituted phenyl, the substituents being selected from the group consisting of hydroxyl, Halogen, Nitro, Cyano, C₁₋₄ Alkyl, C₁₋₄ Alkoxy, C₁₋₄ Alkoxyalkyl, C₁₋₄ Alkoxycarbonyl, C₁₋₄ Alkoxycarbonylalkyl, C₁₋₄ Halogenalkyl, C₆₋₁₀ Aryl, Halogen-C₆₋₁₀ Aryl, C₆₋₁₀ Aryloxy, C₆₋₁₀-Aryl-C₁₋₄ alkyl; unsubstituted or substituted heterocycle having 3-11 ring atoms and 1-4 hetero atoms, the hetero atoms being selected from the group consisting of N, O, S, the substituents being selected from the group consisting of Oxo (═O), Halogen, Nitro, Cyano, C₁₋₄ Alkyl, C₁₋₄ Alkoxy, C₁₋₄ Alkoxyalkyl, C₁₋₄ Alkoxycarbonyl, C₁₋₄ Alkoxycarbonylalkyl, C₁₋₄ Halogenalkyl, C₆₋₁₀ Aryl, Halogen-C₆₋₁₀ Aryl, C₆₋₁₀ Aryloxy, C₆₋₁₀-Aryl-C₁₋₄ alkyl.
R² further preferably represents CH₂R⁶, SR⁶, S(O)R⁶, S(O)₂R⁶ wherein R⁶ represents an unsubstituted or substituted heterocycle having 3-11 ring atoms and 1-4 hetero atoms; the hetero atoms being selected from the group consisting of N, O, S, the substituents being selected from the group consisting of Oxo (═O), Halogen, Nitro, Cyano, C₁₋₄ Alkyl, C₁₋₄ Alkoxy, C₁₋₄ Alkoxyalkyl, C₁₋₄ Alkoxycarbonyl, C₁₋₄ Alkoxycarbonylalkyl, C₁₋₄ Halogenalkyl, C₆₋₁₀ Aryl, Halogen-β6-10 Aryl, C₆₋₁₀ Aryloxy, C₆₋₁₀-Aryl-C₁₋₄ alkyl.
R² particularly preferably represents an unsubstituted, a single or twofold substituted heterocycle having 5-9 ring atoms and 1-3 hetero atoms; the hetero atoms being selected from the group consisting of N, O; the substituents being selected from the group consisting of Halogen, C₁₋₄ Alkyl, C₁₋₄ Alkoxy, C₆₋₁₀ Aryl, Halogen-C₆₋₁₀ Aryl, C₆₋₁₀ Aryloxy, C₆₋₁₀-Aryl-C₁₋₄ alkyl.
R² particularly preferably represents phenyl, substituted by one or two substituents, the substituents being selected from the group consisting of Halogen, Cyano, C₁₋₄ Alkyl, C₁₋₄ Alkoxy, C₆₋₁₀ Aryl, Halogen-C₆₋₁₀ Aryl, C₆₋₁₀ Aryloxy, C₆₋₁₀-Aryl-C₁₋₄ alkyl.
R² further particularly preferably represents C(O)R³ wherein R³ represents C₁₋₄ alkyl; C₁₋₄ alkoxy or substituted C₁₋₄ alkoxy, the substituents being selected from the group consisting of chlorophenyl, bromophenyl, trifluoromethylphenyl, methoxyphenyl; phenyl or substituted phenyl, the substituents being selected from the group consisting of Halogen, C₁₋₄ Alkyl, C₁₋₄ Alkoxy, C₆₋₁₀ Aryl, Halogen-C₆₋₁₀ Aryl, C₆₋₁₀ Aryloxy, C₆₋₁₀-Aryl-C₁₋₄ alkyl; an unsubstituted, a single or twofold substituted heterocycle having 5-9 ring atoms and 1-3 hetero atoms, the hetero atoms being selected from the group consisting of N, O; the substituents being selected from the group consisting of Halogen, C₁₋₄ Alkyl, C₁₋₄ Alkoxy, C₆₋₁₀ Aryl, Halogen-C₆₋₁₀ Aryl, C₆₋₁₀ Aryloxy, C₆₋₁₀-Aryl-C₁₋₄ alkyl.
R² further particularly preferably represents C(O)R³ wherein R³ represents unsubstituted C₃₋₁₂ cycloalkyl or substituted C₃₋₁₂ cycloalkyl, the substituents being selected from the group consisting of Halogen, C₁₋₄ Alkyl, C₁₋₄ Alkoxy, C₁₋₄ Alkylcarbonyl, C₁₋₄ Alkoxycarbonyl.
R² further particularly preferably represents CH₂R⁶, SR⁶, S(O)R⁶, S(O)₂R⁶ wherein R⁶ represents an unsubstituted or substituted heterocycle having 3-11 ring atoms and 1-4 hetero atoms; the hetero atoms being selected from the group consisting of N, O, S, the substituents being selected from the group consisting of Oxo (═O), Halogen, Nitro, Cyano, C₁₋₄ Alkyl, C₁₋₄ Alkoxy, C₁₋₄ Alkoxyalkyl, C₁₋₄ Alkoxycarbonyl, C₁₋₄ Alkoxycarbonylalkyl, C₁₋₄ Halogenalkyl, C₆₋₁₀ Aryl, Halogen-C₆₋₁₀ Aryl, C₆₋₁₀ Aryloxy, C₆₋₁₀-Aryl-C₁₋₄ alkyl.
R² very particularly preferably represents one of the following:

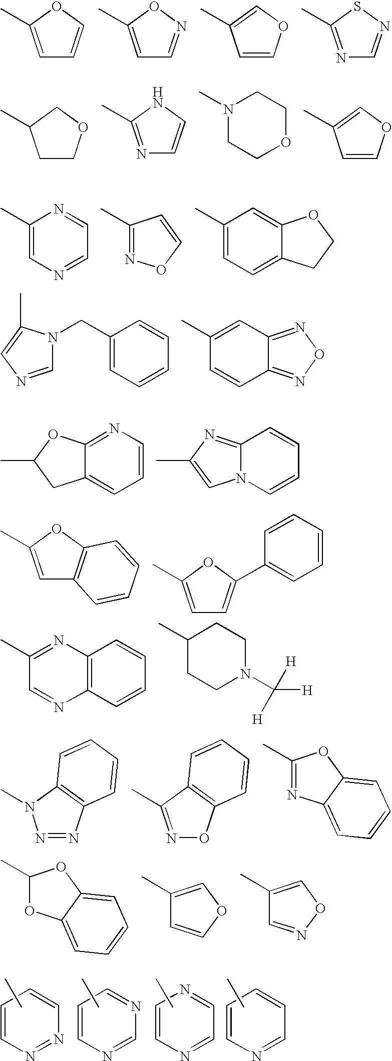

and the substituents selected from the group consisting of fluoro, chloro, methyl, tert.butyl, methoxy, methylthio, difluormethyl, trifluormethyl, amino.
R² particularly preferably represents phenyl, substituted by one or two substituents, the substituents being selected from the group consisting of fluoro, chloro, cyano, methyl, ethyl, n-propyl, iso-propyl, methoxy, ethoxy, n-propoxy, i-propoxy.
R² further very particularly preferably represents C(O)R³ wherein R³ represents methyl, ethyl, n- or iso-propyl, n-, iso-, sec- or tert-butyl, methoxy, ethoxy, n- or iso-propoxy, n-, iso-, sec- or tert-butoxy or one of the following heterocycles:

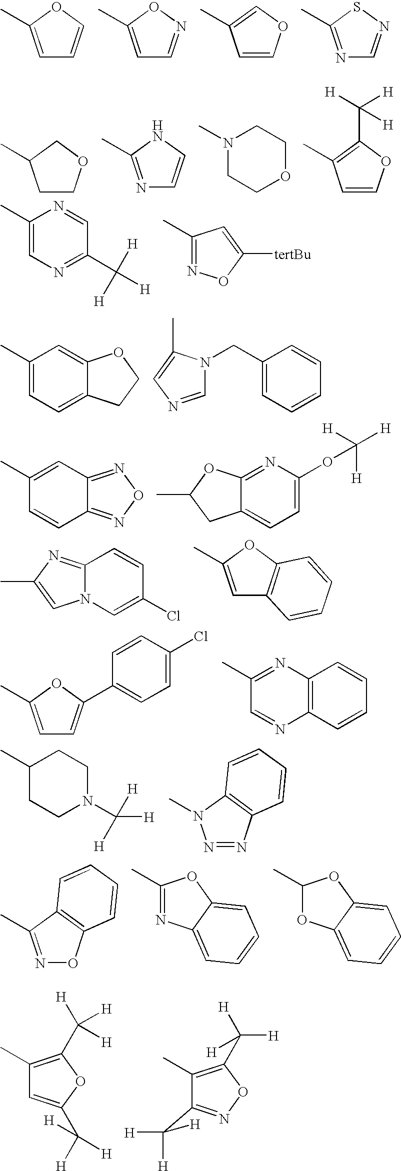

R² further very particularly preferably represents CH₂R⁶, SR⁶, S(O)R⁶, S(O)₂R⁶ wherein R⁶ represents one of the following heterocycles:

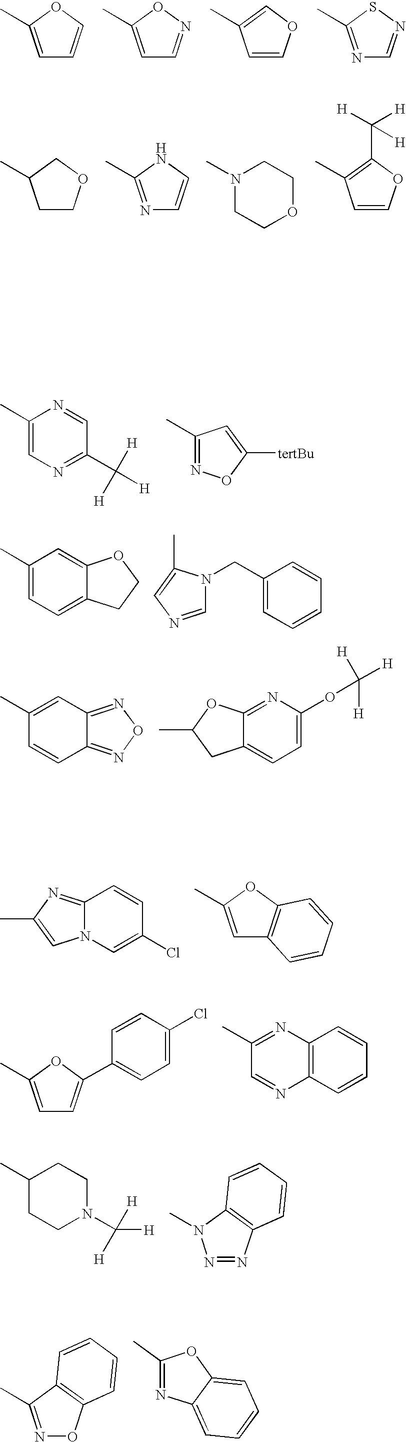

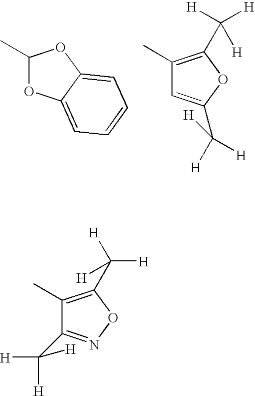

X² preferably represents C₁₋₆ alkandiyl, C₁₋₆ alkandiyl with an oxygen group at the end or C₁₋₆ alkandiyl with an carbonyl group at the end, C₁₋₆ alkandiyl with an carbonyloxy group at the end.
X² particular preferably represents, methandiyl (—CH₂—), 1,2-ethanediyl (—CH₂—CH₂—), 1,1-ethanediyl ((—CH(CH₃)—), 1,3-propanediyl, methandiyloxy (—O—CH₂—), 1,2-ethanediyloxy (—O—CH₂—CH₂—), 1,1-ethanediyloxy ((—O—CH(CH₃)—), methandiylcarbonyl (—CO—CH₂—), 1,2-ethanediylcarbonyl (—CO—CH₂—CH₂—), 1,1-ethanediylcarbonyl ((—CO—CH(CH₃)—), methandiylcarbonyloxy (—C(O)O—CH₂—), 1,2-ethanediylcarbonyloxy (—C(O)O—CH₂—CH₂—), 1,1-ethanediylcarbonyloxy ((—C(O)O—CH(CH₃)—). The functional groups as defined for X are preferably bound to the group R².

The abovementioned general or preferred radical definitions apply both to the end products of the formula (I) and also, correspondingly, to the starting materials or intermediates required in each case for the preparation. These radical definitions can be combined with one another at will, i.e. including combinations between the given preferred ranges. Further, individual definitions may not apply.

Preference according to the invention is given to compounds of the formula (I) which contain a combination of the meanings mentioned above as being preferred.

Particular preference according to the invention is given to compounds of the formula (I) which contain a combination of the meanings listed above as being particularly preferred.

Very particular preference according to the invention is given to the compounds of the formula (I) which contain a combination of the meanings listed above as being very particularly preferred.

In a further aspect, the invention provides compounds of formula (I′)

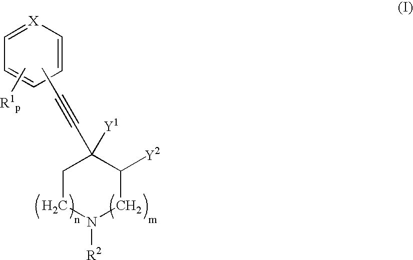

wherein
m represents 0 and n represents 1 or
m represents 0 and n represents 2 or
m represents 1 and n represents 1;
p represents 0, 1, 2, 3, 4 or 5;
X represents CH, N;
Y¹ represents OH and Y² represents H or
Y¹ and Y² form a bond;
R¹ represents halogen, cyano, nitro, —CHO, alkyl, alkoxy, halogenalkoxy, halogenalkyl, —C(O)R⁴, —COOR⁴ wherein R⁴ is alkyl or two substituents R¹ together form a alkandiyl or alkenediyl-moiety;
R² represents an unsubstituted or substituted heterocycle, or
R² represents phenyl or substituted phenyl, or
R² represents C(O)R³ wherein R³ represents alkyl, alkoxy or substituted alkoxy, phenyl or substituted phenyl, an unsubstituted or substituted aliphatic heterocycle, an unsubstituted or substituted partly saturated heterocycle containing less than 12 ring atoms, an unsubstituted or substituted aromatic heterocycle containing less than 12 ring atoms or
R² represents CH₂R⁶, SR⁶, S(O)R⁶, S(O)₂R⁶ wherein R⁶ represents an unsubstituted or substituted heterocycle
   in free base or acid addition salt form.

A preferred group of compounds of formula (I) is represented by formula (I-I)

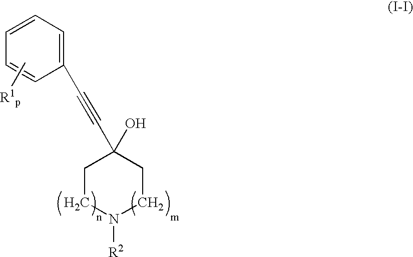

wherein R¹, R², m, n and p are as defined above.

A further preferred group of compounds of formula (I) is represented by formula (I-II)

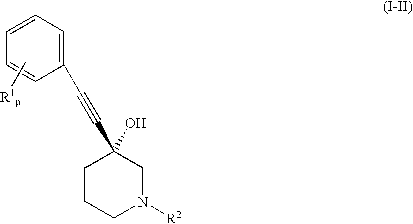

wherein R¹, R² and p are as defined above.

A further preferred group of compounds of formula (I) is represented by formula (I-III)

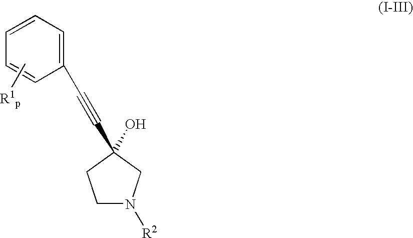

wherein R¹, R² and p are as defined above.

A further preferred group of compounds of formula (I) is represented by formula (I-IV)

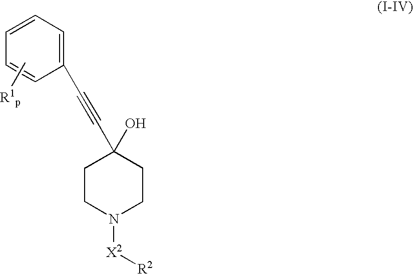

wherein X², R¹ and p are as defined above; R² represents phenyl or substituted phenyl.

A further preferred group of compounds of formula (I) are compounds wherein o represents 1, X represents CH and, R¹ is in the meta-position.

In a further aspect, the invention provides processes for the production of the compounds of formula (I) and their salts as well as their starting materials.

A first process for the production of the compounds of formula (I) and their salts, comprises the steps of

i) reacting a compound of formula (II)

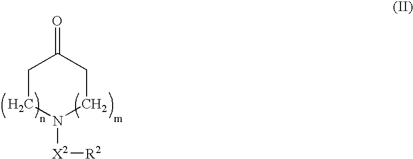

wherein X², R², m, n are as defined above, with a compound of formula (III)

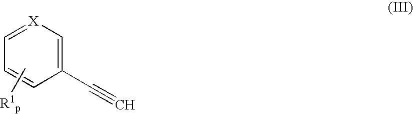

wherein R¹, X and p are as defined above, in the presence of a base, resulting in compounds of formula (I) wherein Y¹ represents OH and Y² represents H; or
ii) - in case X² represents a single bond - reacting a compound of formula (IV)

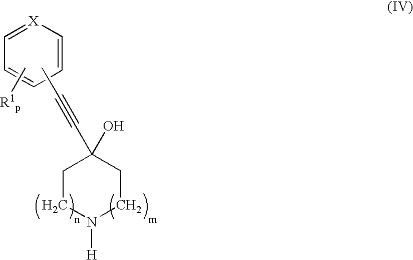

wherein R¹, X, m, n and p are as defined above, with a compound of formula (V)

LG-R² (V)

wherein R² is as defined above and LG represents a leaving group, e.g. a halogen such as Br or Cl, optionally in the presence of reaction auxiliaries, optionally in the presence of a diluent; or
iii) - in case X² represents a single bond - reacting a compound of formula (IV)

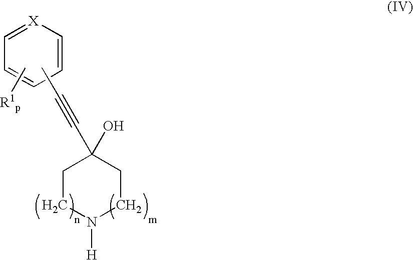

wherein R¹, X, m, n and p are as defined above, with a compound of formula (VI)

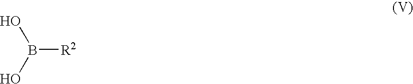

wherein R² is as defined above, optionally in the presence of reaction auxiliaries, optionally in the presence of a diluent; or
iv) reacting a compound of formula (IV) wherein R¹, X, m, n and p are as defined above by reductive amination with a compound of formula (VII)

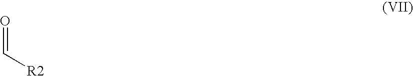

wherein R² is defined as above, or
v) - in case represents carbonyl - reacting a compound of formula (IV)

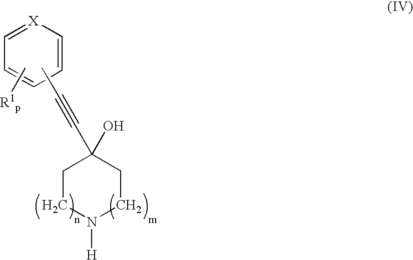

wherein R¹, X, m, n and p are as defined above, with a compound of formula (IIX)

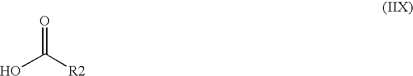

wherein R² is defined as above, optionally in the presence of reaction auxiliaries, optionally in the presence of a diluent and
vi) optionally converting the substituent X²—R² into another substituent X²—R² according to conventional procedures; and
vii) optionally eliminating H₂O from the thus obtained compound resulting in a compound of formula (I) wherein Y¹ and Y² form a bond and
viii) recovering the resulting compound of formula (I) in free base or acid addition salt form.

The reaction steps of Process 1 are described in more detail hereinafter:

Step i) The starting materials of formulae (II) and (III) are known or may be obtained from known compounds, using conventional procedures.

For performing step i), a compound of formula (III), optionally diluted in a diluent, such as THF, is treated with a base, e.g. BuLi, preferably 0.8 to 1.2 equivalents, most preferably in equimolar amounts at low temperatures, e.g. at −75° C. To this reaction mixture is added a compound of formula (II), optionally diluted in a diluent, such as THF, at low temperatures, e.g. −75° C. to 0° C., preferably −75° C. to −55° C. The reaction mixture is than extracted at ambient temperature using e.g. H₂O/MTBE. After purification, e.g. crystallization from a second solvent, for example Et₂O/hexane, the compound of formula (I) is obtained. If necessary, protected moieties such as hydroxyl or amino functions within the reaction product can be deprotected; the reaction product may be further converted, e.g. by substitution, elimination, reduction or oxidation reaction.

Step ii) This reaction is known as “Buchwald-Hartwig reaction” typical reaction conditions and auxiliaries are used. The starting materials of formula (V) are known or may be obtained from known compounds, using conventional procedures; the starting material of formula (IV) is new and may be obtained according to process 2, described below.

A leaving group LG represents any moiety that may be replaced under reaction conditions to yield compounds of formula (I). Such leaving groups are known to the expert and include, for example, halogen-, tosyl- and Protecting groups.

Step iii): The starting materials of formula (V) are known or may be obtained according to known procedures. Typically reaction auxiliaries, such as organic copper compounds may be employed.

Step iv): This reaction step may be regarded as a reductive amination. The starting materials of formula (VII) are known or may be obtained according to known procedures. Typical reaction auxiliaries are reductive agents, such as Hydrides, e.g. Sodium-triacetoxyborohydride.

Step v): For performing step v), a mixture of compound (IV) and compound (IIX) e.g. in equimolar amounts, neat or dissolved in a suitable inert solvent, such as dmf, are treated with a base, e.g. Et₃N, preferably 1 to 2 equivalents, most preferably 1.2 to 1.5 equivalents, and reaction auxiliaries, such as HOBt and EDC preferably 1 to 2 equivalents, most preferably 1.2 to 1.5 equivalents each, for a longer period of time, e.g. 1 to 24 h, at low temperatures, e.g. −10° C. to r.t. If necessary, protected moieties such as hydroxyl or amino functions within the reaction product can be deprotected; the reaction product may be further converted, e.g. by oxidation reaction; the reaction product may be purified according to conventional methods, e.g. by column chromatography or recrystallisation.

The following reaction scheme is illustrative for step v)

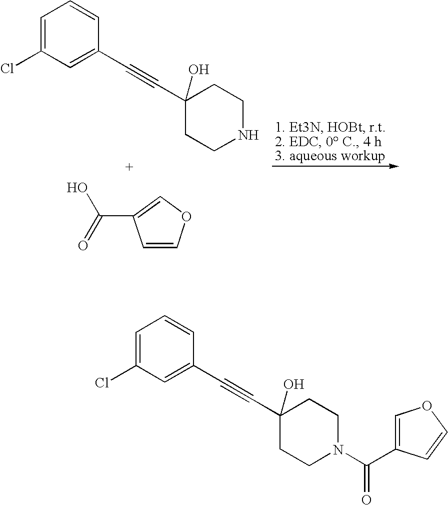

Step vi) Compounds of formula (I) obtained in accordance with the above-described process can be converted into other compounds of formula (I) in customary manner, e.g by substitution, elimination, addition, reduction or oxidation reactions.

Step vii) By eliminating the hydroxy-group Y¹ of compounds of formula (I), a C═C double bond may be formed. For example, a compound of formula (I-I), in the presence of a base and in the presence of a solvent, may be subject to a reaction with POCl₃ and be isolated after aqueous work-up resulting in a compound of formula (I) wherein Y¹ and Y² represent a bond.

For performing step vii), a mixture of a compound of formula (I) wherein Y¹ represents OH and Y² represents H, and a base, such as Et₃N, preferably 1 to 20 equivalents, most preferably 5 to 15 equivalents, optionally diluted in an inert diluent, such as DCM, is treated with POCl₃, preferably 1 to 10 equivalents, most preferably 1.5 to 3 equivalents at r.t. and reacted for a longer period of time, preferably 1 to 24 hours, e.g. 15 hours. The reaction product obtained is poured into aqueous base, e.g. NaOH/H₂O, extracted with a suitable solvent, e.g. EtOAc and purified e.g. by chromatography.

Step viii) Working up the reaction mixtures according to the above processes and purification of the compounds thus obtained may be carried out in accordance to known procedures. This includes recrystallisation, salt-formation and purification via column chromatography. Acid addition salts may be produced from the free bases in known manner, and vice versa. Resulting acid addition salts can be converted into other acid addition salts or into the free bases in a manner known per se. The compounds of formula (I), including their acid addition salts, may also be obtained in the form of hydrates or may include the solvent used for crystallization.

In a further aspect of the invention, compounds of formula (IV)

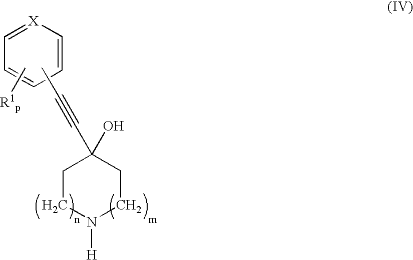

wherein R¹, X, m, n, p are as defined above and their acid addition salts, which are useful as intermediates for the manufacture of compounds of formula (I), are provided.

Compounds of formula (IV) may be obtained according to Process 2, which comprises the step of reacting a compound of formula (III)

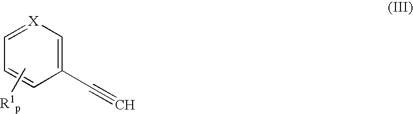

wherein R¹ and X are as defined above with a compound of formula (VI)

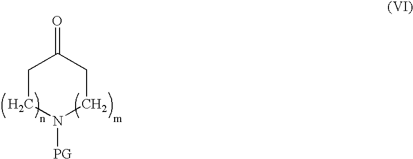

wherein m and n are as defined above and PG represents a protecting group, in the presence of a base, optionally in the presence of a diluent.

The reaction steps of Process 2 are described in more detail hereinafter:

A suitable protecting group PG is any protecting group which is stable under basic conditions, for example the Cbo-, Fmoc- or BOC group, preferably the BOC-group.

A suitable base is any base capable for deprotonation a compound of formula (III) at the triple bond, for example an alkalimetalhydrid, an earthalkylimetalhydrid, an alkalimetalalkyle, an earthalkylimetalalkyle, preferably an alkalimetalalkyle, e.g. Butyllithium.

The reaction may take place in the presence of a diluent. Suitable diluents are inert under reaction conditions, for example alkanes, e.g. hexane, or cyclohexane, ethers, e.g. diethylether or thf, or mixtures of such diluents.

For performing Process 2), a compound of formula (III), optionally diluted in a diluent, such as thf, is treated with a base, e.g. BuLi, preferably 0.8 to 1.2 equivalents, most preferably in equimolar amounts at low temperatures, e.g. at −75° C. To this reaction mixture is added a compound of formula (VI), optionally diluted in a diluent, such as thf, at low temperatures, e.g. −75° C. to 0° C., preferably −75° C. to −55° C. The reaction mixture is than extracted at ambient temperature using e.g H₂O/MTBE. Deprotection is accomplished by dissolving the crude product in an inert solvent, e.g. EtOAC and adding an acid, e.g. HCl in dioxane, in excess, e.g. 1.5 to 15 equivalents, at low temperatures, e.g. 0° C. The reaction mixture is poured into aqueous alkaline solution, e.g. H₂O/K₂CO₃, and extracted with a suitable solvent, e.g. EtOAc. After purification, e.g. crystallization from a second solvent, for example Et₂O/hexane, the compound of formula (IV) is obtained. Alternatively, the product may directly used for further reaction steps without purification.

The following reaction scheme is illustrative for Process 2):

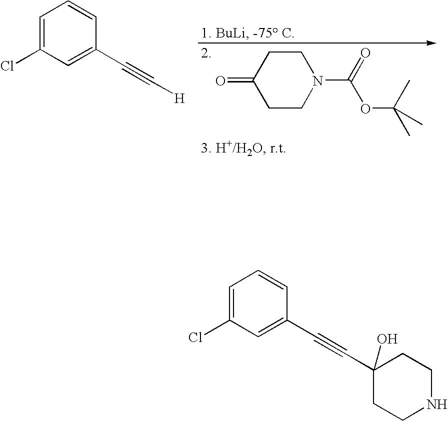

The following considerations apply to the individual reaction steps described in process 1 and 2:

a) One or more functional groups, for example carboxy, hydroxy, amino, or mercapto, may need to be protected in the starting materials by protecting groups. The protecting groups employed may already be present in precursors and should protect the functional groups concerned against unwanted secondary reactions, such as acylations, etherifications, esterifications, oxidations, solvolysis, and similar reactions. It is a characteristic of protecting groups that they lend themselves readily, i.e. without undesired secondary reactions, to removal, typically by solvolysis, reduction, photolysis or also by enzyme activity, for example under conditions analogous to physiological conditions, and that they are not present in the end-products. The specialist knows, or can easily establish, which protecting groups are suitable with the reactions mentioned hereinabove and hereinafter. The protection of such functional groups by such protecting groups, the protecting groups themselves, and their removal reactions are described for example in standard reference works, such as J. F. W. McOmie, “Protective Groups in Organic Chemistry”, Plenum Press, London and New York 1973, in T. W. Greene, “Protective Groups in Organic Synthesis”, Wiley, New York 1981, in “The Peptides”; Volume 3 (editors: E. Gross and J. Meienhofer), Academic Press, London and New York 1981, in “Methoden der organischen Chemie” (Methods of organic chemistry), Houben Weyl, 4th edition, Volume 15/l, Georg Thieme Verlag, Stuttgart 1974, in H.-D. Jakubke and H. Jescheit, “Aminosauren, Peptide, Proteine” (Amino acids, peptides, proteins), Verlag Chemie, Weinheim, Deerfield Beach, and Basel 1982, and in Jochen Lehmann, “Chemie der Kohlenhydrate: Monosaccharide und Derivate” (Chemistry of carbohydrates: monosaccharides and derivatives), Georg Thieme Verlag, Stuttgart 1974.

b) Acid addition salts may be produced from the free bases in known manner, and vice-versa. Compounds of formula (I) in optically pure form can be obtained from the corresponding racemates according to well-known procedures, e.g. HPLC with chiral matrix. Alternatively, optically pure starting materials can be used.

c) Stereoisomeric mixtures, e.g. mixtures of diastereomers, can be separated into their corresponding isomers in a manner known per se by means of suitable separation methods. Diastereomeric mixtures for example may be separated into their individual diastereomers by means of fractionated crystallization, chromatography, solvent distribution, and similar procedures. This separation may take place either at the level of a starting compound or in a compound of formula I itself. Enantiomers may be separated through the formation of diastereomeric salts, for example by salt formation with an enantiomer-pure chiral acid, or by means of chromatography, for example by HPLC, using chromatographic substrates with chiral ligands.

d) Suitable diluents for carrying out the above-described are especially inert organic solvents. These include, in particular, aliphatic, alicyclic or aromatic, optionally halogenated hydrocarbons, such as, for example, benzine, benzene, toluene, xylene, chlorobenzene, dichlorobenzene, petroleum ether, hexane, cyclohexane, dichloromethane, chloroform, carbon tetrachloride; ethers, such as diethyl ether, diisopropyl ether, dioxane, tetrahydrofuran or ethylene glycol dimethyl ether or ethylene glycol diethyl ether; ketones, such as acetone, butanone or methyl isobutyl ketone; nitriles, such as acetonitrile propionitrile or butyronitrile; amides, such as N,N-dimethylformamide, N,N-dimethylacetamide, N-methyl-formanilide, N-methyl-pyrrolidone or hexamethylphosphoric triamide; esters, such as methyl acetate or ethyl acetate, sulphoxides, such as dimethyl sulphoxide, alcohols, such as methanol, ethanol, n- or i-propanol, ethylene glycol monomethyl ether, ethylene glycol monoethyl ether, diethyelene glycol monomethyl ether, diethylene glycol monoethyl ether. Further, mixtures of diluents may be employed. Depending on the starting materials, reaction conditions and auxiliaries, water or diluents constaining water may be suitable. It is also possible to use one a starting material as diluent simultaneously.

e) Reaction temperatures can be varied within a relatively wide range. In general, the processes are carried out at temperatures between 0° C. and 150° C., preferably between 10° C. and 120° C. Deprotonation reactions can be varied within a relatively wide range. In general, the processes are carried out at temperatures between −150° C. and +50° C., preferably between −75° C. and 0° C.

f) The reactions are generally carried out under atmospheric pressure. However, it is also possible to carry out the processes according to the invention under elevated or reduced pressure—in general between 0.1 bar and 10 bar.

g) Starting materials are generally employed in approximately equimolar amounts. However, it is also possible to use a relatively large excess of one of the components. The reaction is generally carried out in a suitable diluent in the presence of a reaction auxiliary, and the reaction mixture is generally stirred at the required temperature for a number of hours.

h) Work-up is carried out by customary methods (cf. the Preparation Examples).

Compounds of formula (I) and their pharmaceutically acceptable acid addition salts, hereinafter referred to as agents of the invention, exhibit valuable pharmacological properties and are therefore useful as pharmaceuticals.

In particular, the agents of the invention exhibit a marked and selective modulating, especially antagonistic, action at human metabotropic glutamate receptors (mGluRs). This can be determined in vitro for example at recombinant human metabotropic glutamate receptors, especially PLC-coupled subtypes thereof such as mGluRs, using different procedures like, for example, measurement of the inhibition of the agonist induced elevation of intracellular Ca²⁺ concentration in accordance with L. P. Daggett et al., Neuropharm. Vol. 34, pages 871-886 (1995), P. J. Flor et al., J. Neurochem. Vol. 67, pages 58-63 (1996) or by determination to what extent the agonist induced elevation of the inositol phosphate turnover is inhibited as described by T. Knoepfel et al., Eur. J. Pharmacol. Vol. 288, pages 389-392 (1994), L. P. Daggett et al., Neuropharm. Vol. 67, pages 58-63 (1996) and references cited therein. Isolation and expression of human mGluR subtypes are described in U.S. Pat. No. 5,521,297. Selected agents of the invention show IC50 values for the inhibition of the agonist (e.g. glutamate or quisqualate) induced elevation of intracellular Ca2+ concentration or the agonist (e.g. glutamate or quisqualate) induced inositol phosphate turnover, measured in recombinant cells expressing hmGluR5a of about 1 nM to about 50 μM.

The agents of the invention are therefore useful in the prevention, treatment or delay of progression of disorders associated with irregularities of the glutamatergic signal transmission, of the gastro-intestinal and urinary tract and of nervous system disorders mediated full or in part by mGluR5.

Disorders associated with irregularities of the glutamatergic signal transmission are for example epilepsy, cerebral ischemias, especially acute ischemias, ischemic diseases of the eye, muscle spasms such as local or general spasticity, skin disorders, obesity disorders and, in particular, convulsions or pain.

Disorders of the gastro-intestinal tract include post-operative ileus, functional gastro-intestinal disorders (FGID) as for example functional dyspepsia (FD), gastro-esophageal reflux disease (GERD), irritable bowel syndrome (IBS), functional bloating, functional diarrhea, chronic constipation, functional disturbancies of the biliary tract as well as other conditions according to Gut 1999; Vol. 45 Suppl. II.

Disorders of the Urinary Tract comprise conditions associated with pain and/or discomfort of the urinary tract and overactive bladder (OAB).

Nervous system disorders mediated full or in part by mGluR5 are for example acute, traumatic and chronic degenerative processes of the nervous system, such as Parkinson's disease, senile dementia, Alzheimer's disease, Huntington's chorea, amyotrophic lateral sclerosis, multiple sclerosis and fragile X syndrome, psychiatric diseases such as schizophrenia and anxiety, depression, pain, itch and drug abuse. Anxiety related disorders includes panic disorders, social anxiety, obsessive compulsive disorders (OCD), post traumatic stress disorders (ATSD), generalized anxiety disorders (GAD), phobias.

The usefulness of the agents of the invention in the treatment of the above-mentioned disorders can be confirmed in a range of standard tests including those indicated below: Activity of the agents of the invention in anxiety can be demonstrated in standard models such as the stress-induced hyperthermia in mice [cf. A. Lecci et al., Psychopharmacol 101, 255-261]. At doses of about 0.1 to about 30 mg/kg p.o., selected agents of the invention reverse the stress-induced hyperthermia.

At doses of about 4 to about 50 mg/kg p.o., selected agents of the invention show reversal of Freund complete adjuvant (FCA) induced hyperalgesia [cf. J. Donnerer et al., Neuroscience 49, 693-698 (1992) and C. J. Woolf, Neuroscience 62, 327-331 (1994)].

For all the above mentioned indications, the appropriate dosage will of course vary depending upon, for example, the compound employed, the host, the mode of administration and the nature and severity of the condition being treated. However, in general, satisfactory results in animals are indicated to be obtained at a daily dosage of from about 0.5 to about 100 mg/kg animal body weight. In larger mammals, for example humans, an indicated daily dosage is in the range from about 5 to 1500 mg, preferably about 10 to about 1000 mg of the compound conveniently administered in divided doses up to 4 times a day or in sustained release form.

In accordance with the foregoing, the present invention also provides an agent of the invention for use as a pharmaceutical, e.g. in the prevention, treatment or delay of progression of disorders associated with irregularities of the glutamatergic signal transmission, of the gastrointestinal and urinary tract and of nervous system disorders mediated full or in part by mGluR5.

The invention also provides the use of an agent of the invention, in the prevention, treatment or delay of progression of disorders associated with irregularities of the glutamatergic signal transmission, of the gastrointestinal and urinary tract and of nervous system disorders mediated full or in part by mGluR5.

Furthermore the invention provides the use of an agent of the invention for the manufacture of a pharmaceutical composition designed for the prevention, treatment or delay of progression of disorders associated with irregularities of the glutamatergic signal transmission, of the gastrointestinal and urinary tract and of nervous system disorders mediated full or in part by mGluR5.

In a further aspect the invention relates to a method of treating disorders mediated full or in part by mGluR5, which method comprises administering to a warm-blooded organism in need of such treatment a therapeutically effective amount of an agent of the invention.

Moreover the invention relates to a pharmaceutical composition comprising an agent of the invention in association with one or more pharmaceutical carrier or one or more pharmaceutically acceptable diluent.

The pharmaceutical compositions according to the invention are compositions for enteral, such as nasal, rectal or oral, or parenteral, such as intramuscular or intravenous, administration to warm-blooded animals (human beings and animals) that comprise an effective dose of the pharmacological active ingredient alone or together with a significant amount of a pharmaceutically acceptable carrier. The dose of the active ingredient depends on the species of warm-blooded animal, body weight, age and individual condition, individual pharmacokinetic data, the disease to be treated and the mode of administration.

The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, dragées, tablets or capsules.

The pharmaceutical compositions of the present invention are prepared in a manner known per se, for example by means of conventional dissolving, lyophilizing, mixing, granulating or confectioning processes.

The preferred agents of the invention include the Benzofuran-2-yl-[4-(3-chloro-phenyl-ethynyl)-4-hydroxy-piperidin-1-yl]-methanone free base or pharmaceutically acceptable acid addition salt form.

Said compound Benzofuran-2-yl-[4-(3-chloro-phenyl-ethynyl)-4-hydroxy-piperidin-1-yl]-methanone inhibits the quinqualate-induced inositol phosphate turnover in hmGluR5 expressing cells with an IC₅₀ concentration of 290 nM.

With the same compound, a stress-induced hyperthermia of 0.93±0.1° C. was reduced to 0.44±0.08° C. at 10 mg/kg p.o., to 0.46±0.14° C. at 30 mg/kg p.o. and to 0.24±0.12° C. at 100 mg/kg p.o. (p<0.01; p<0.05; p<0.001 respectively).

Further, properly isotope-labeled agents of the invention exhibit valuable properties as histopathological labeling agents, imaging agents and/or biomarkers, hereinafter “markers”, for the selective labeling of the metabotropic glutamate receptor subtype 5 (mGlu5 receptor). More particularly the agents of the invention are useful as markers for labeling the central and peripheral mGlu5 receptors in vitro or in vivo. In particular, compounds of the invention which are properly isotopically labeled are useful as PET markers. Such PET markers are labeled with one or more atoms selected from the group consisting of ¹¹C, ¹³N, ¹⁵O, ¹⁸F.

The agents of the invention are therefore useful, for instance, for determining the levels of receptor occupancy of a drug acting at the mGlu5 receptor, or diagnostic purposes for diseases resulting from an imbalance or dysfunction of mGlu5 receptors, and for monitoring the effectiveness of pharmacotherapies of such diseases.

In accordance with the above, the present invention provides an agent of the invention for use as a marker for neuroimaging.

In a further aspect, the present invention provides a composition for labeling brain and peripheral nervous system structures involving mGlu5 receptors in vivo and in vitro comprising an agent of the invention.

In still a further aspect, the present invention provides a method for labeling brain and peripheral nervous system structures involving mGlu5 receptors in vitro or in vivo, which comprises contacting brain tissue with an agent of the invention.

The method of the invention may comprise a further step aimed at determining whether the agent of the invention labeled the target structure. Said further step may be effected by observing the target structure using positron emission tomography (PET) or single photon emission computed tomography (SPECT), or any device allowing detection of radioactive radiations.

The following non-limiting Examples illustrate the invention. A list of Abbreviations used is given below.

BOC tert-butoxycarbonyl
n-BuLi n-butyl lithium
DCM dichloromethane
DMF N,N′-dimethylformamide
EDC 1-ethyl-3-[3-(dimethylamino)propyl]-carbodiimide hydrochloride
EtOAc ethylacetate
h hours
HCl hydrochloric acid
HOBt hydroxybenzotriazole
HPLC high pressure liquid chromatography
min minutes
Mp melting point
MS mass spectroscopy
MTBE methyl-tert.-butylether
Rf retention factor (Thin Layer Chromatography)
Rt retention time (LC/MS)
rt room temperature
TFA trifluoroacetic acid
THF tetrahydrofuran

### EXAMPLE 1

### [4-(3-Chloro-phenylethynyl)-4-hydroxy-piperidin-1-yl]-furan-3-yl-methanone

A solution of 4-(3-Chloro-phenylethynyl)-piperidin-4-ol (0.707 g, 3 mmol) and furane-3-carboxylic acid (0.403 g, 3.6 mmol) in DMF (12 ml) was treated with Et₃N (0.501 ml, 3.6 mmol) and HOBt (0.405 g, 3 mmol), and cooled to 0° C. EDC (0.690 g, 3.6 mmol) was added and the ice bath was removed. After stirring for 4 h, 2M NaHCO₃ (100 ml) was added and the mixture was extracted with DCM (2×100 ml). The combined extracts were washed with 0.5 M citric acid (1×100 ml) and brine (1×100 ml) and dried over Na₂SO₄. Filtration and evaporation of the solvents afforded a yellowish oil (1.03 g). Chromatography on SiO₂ (EtOAc/cyclohexanol 1:1) afforded a colorless oil which was crystallized from Et₂O/hexane which led to the title compound as white crystals (0.645 g, 65%).

Mp: 93-94° C.;

MS (LC/MS): 330.3 [M+H];

TLC Rf: 0.49 (EtOAc).

The starting material was prepared as described hereafter:

### 4-(3-Chloro-phenylethynyl)-piperidin-4-ol

A solution of 1-Chloro-3-ethynyl-benzene (11.86 g, 86.8 mmol) in THF (200 ml) was cooled to −75° C. Within 30 minutes, a solution of n-BuLi in hexane (1.5 N, 58 ml, 87 mmol) was added and the mixture stirred for 30 minutes at −75° C. A solution of 4-oxo-piperidine-1-carboxylic acid tert-butyl ester (17.3 g, 86.8 mmol) in THF (100 ml) was added dropwise within 45 minutes at −75° C. The cooling bath was removed, and when the mixture had reached room temperature it was slowly poured into a stirred mixture of ice water (1000 ml) and MTBE (500 ml). The aqueous phase was separated and extracted with MTBE (250 ml). The combined organic phases were washed with water (250 ml), dried over Na₂SO₄, filtered and the solvent evaporated to afford 4-(3-chloro-phenylethynyl)-4-hydroxy-piperidine-1-carboxylic acid tert-butyl ester as a yellowish oil (30.0 g, 100%) that was used without further purification. This Boc-protected amine (4.1 g, 12.2 mmol) was dissolved in EtOAc (40 ml) and cooled to 0° C. A 4 N solution of HCl in dioxane (37.5 ml, 150 mmol) was added in portions. After stirring this mixture for a total of 2 h at 0° C., it was poured into a 2N aqueous solution of K₂CO₃ (75 ml). The aqueous phase was separated and extracted with EtOAc (25 ml). The combined organic phases were dried over Na₂SO₄, filtered and the solvent evaporated. Chromatography of the residue afforded 4-(3-chloro-phenylethynyl)-piperidin-4-ol (1.23 g, 43%) as a brownish foam. Crystallization from Et₂O/hexane yielded yellow-brownish crystals.

M.p. 95-103° C.

Following the same procedure, the following compounds can be obtained:

### EXAMPLE 1.1

### [4-(3-Chloro-phenylethynyl)-4-hydroxy-piperidin-1-yl]-(tetrahydro-furan-3-yl)-methanone

MS (LC/MS): 334 [M+H]

TLC Rf: 0.36 (EtOAc)

### EXAMPLE 1.2

### [4-(3-Chloro-phenylethynyl)-4-hydroxy-piperidin-1-yl]-(1-methyl-piperidin-4-yl)-methanone

TLC Rf: 0.38 (DCM/MeOH/NH₄OH 85:15:1)

Mp: 134-136° C.

### EXAMPLE 1.3

### [4-(3-Chloro-phenylethynyl)-4-hydroxy-piperidin-1-yl]-isoxazol-5-yl-methanone

TLC Rf: 0.55 (DCM/MeOH/NH₄OH 85:15:1)

Mp: 132-135° C.

### EXAMPLE 1.4

### [4-(3-Chloro-phenylethynyl)-4-hydroxy-piperidin-1-yl]-(1H-imidazol-2-yl)-methanone

TLC Rf: 0.31 (DCM/MeOH/NH₄OH 85:15:1)

Mp: 75-80° C.

### EXAMPLE 1.5

### [4-(3-Chloro-phenylethynyl)-4-hydroxy-piperidin-1-yl]-furan-2-yl-methanone

MS (LC/MS): 330 [M+H]

TLC Rf: 0.46 (EtOAc)

### EXAMPLE 1.6

### [4-(3-Chloro-phenylethynyl)-4-hydroxy-piperidin-1-yl]-(5-methyl-pyrazin-2-yl)-methanone

MS (LC/MS): 356 [M+H]

TLC Rf: 0.27 (EtOAc)

### EXAMPLE 1.7

### (6-Chloro-imidazo[1,2-a]pyridin-2-yl)-[4-(3-chloro-phenylethynyl)-4-hydroxy-piperidin-1-yl]-methanone

MS (LC/MS): 415 [M+H]

TLC Rf: 0.60 (EtOAc)

### EXAMPLE 1.8

### Benzofuran-2-yl-[4-(3-chloro-phenyl-ethynyl)-4-hydroxy-piperidin-1-yl]-methanone

MS (LC/MS): 380 [M+H]

TLC Rf: 0.33 (EtOAc)

### EXAMPLE 1.9

### Furan-3-yl-(4-hydroxy-4-isoquinolin-4-ylethynyl-piperidin-1-yl)-methanone

MS (LC/MS): 347 [M+H]

TLC Rf: 0.16 (EtOAc)

### EXAMPLE 1.10

### (3-Benzyl-3H-imidazol-4-yl)-[4-(3-chloro-phenylethynyl)-4-hydroxy-piperidin-1-yl]-methanone

MS (LC/MS): 420 [M+H]

TLC Rf: 0.74 (DCM/MeOH/NH₄OH 85:15:1)

### EXAMPLE 1.11

### [4-(3-Chloro-phenylethynyl)-4-hydroxy-piperidin-1-yl]-[5-(4-chloro-phenyl)-furan-2-yl]-methanone

MS (LC/MS): 441 [M+H]

TLC Rf: 0.26 (EtOAc/hex 1:1)

### EXAMPLE 1.12

### [4-(3-Chloro-phenylethynyl)-4-hydroxy-piperidin-1-yl]-(2,3-dihydro-benzofuran-6-yl)-methanone

MS (LC/MS): 382 [M+H]

TLC Rf: 0.29 (EtOAc/cyclohex 1:1)

### EXAMPLE 1.13

### 2-Benzotriazol-1-yl-1-[4-(3-chloro-phenylethynyl)-4-hydroxy-piperidin-1-yl]-ethanone

MS (LC/MS): 395 [M+H]

TLC Rf: 0.26 (EtOAc/cyclohex 1:1)

### EXAMPLE 1.14

### [4-(3-Chloro-phenylethynyl)-4-hydroxy-piperidin-1-yl]-(6-methoxy-furo[2,3-b]pyridin-2-yl)-methanone

MS (LC/MS): 411 [M+H]

TLC Rf: 0.48 (EtOAc/cyclohex 1:1)

### EXAMPLE 1.15

### [4-(3-Chloro-phenylethynyl)-4-hydroxy-piperidin-1-yl]-(2-methyl-furan-3-yl)-methanone

MS (LC/MS): 344 [M+H]

TLC Rf: 0.39 (EtOAc/MeOH 9:1)

### EXAMPLE 1.16

### Benzo[1,2,5]oxadiazol-5-yl-[4-(3-chloro-phenylethynyl)-4-hydroxy-piperidin-1-yl]-methanone

MS (LC/MS): 382 [M+H]

TLC Rf: 0.35 (EtOAc/cyclohex 1:1)

### EXAMPLE 1.17

### [4-(3-Chloro-phenylethynyl)-4-hydroxy-piperidin-1-yl]-(3,5-dimethyl-isoxazol-4-yl)-methanone

MS (LC/MS): 359 [M+H]

TLC Rf: 0.21 (EtOAc/cyclohex 1:1)

### EXAMPLE 1.18

### [4-(3-Chloro-phenylethynyl)-4-hydroxy-piperidin-1-yl]-(5-methyl-isoxazol-3-yl)-methanone

MS (LC/MS): 345 [M+H]

TLC Rf: 0.26 (EtOAc/cyclohex 1:1)

### EXAMPLE 1.19

### [4-(3-Chloro-phenylethynyl)-4-hydroxy-piperidin-1-yl]-(5-methyl-isoxazol-4-yl)-methanone

MS (LC/MS): 345 [M+H]

TLC Rf: 0.17 (EtOAc/cyclohex 1:1)

### EXAMPLE 1.20

### 1-[4-(3-Chloro-phenylethynyl)-4-hydroxy-piperidin-1-yl]-2-(3-methyl-isoxazol-5-yl)-ethanone

MS (LC/MS): 359 [M+H]

TLC Rf: 0.14 (EtOAc/cyclohex 1:1)

### EXAMPLE 1.21

### 2-Benzo[d]isoxazol-3-yl-1-[4-(3-chloro-phenylethynyl)-4-hydroxy-piperidin-1-yl]-ethanone

MS (LC/MS): 395 [M+H]

TLC Rf: 0.33 (EtOAc/cyclohex 1:1)

### EXAMPLE 1.22

### [4-(3-Chloro-phenylethynyl)-4-hydroxy-piperidin-1-yl]-quinoxalin-2-yl-methanone

MS (LC/MS): 392 [M+H]

TLC Rf: 0.24 (EtOAc/cyclohex 1:1)

### EXAMPLE 1.23

### [4-(3-Chloro-phenylethynyl)-4-hydroxy-piperidin-1-yl]-(2,5-dimethyl-4,5-dihydro-furan-3-yl)-methanone

MS (LC/MS): 358 [M+H]

TLC Rf: 0.25 (EtOAc/cyclohex 1:1)

### EXAMPLE 1.24

### Benzooxazol-2-yl-[4-(3-chloro-phenylethynyl)-4-hydroxy-piperidin-1-yl]-methanone

MS (LC/MS): 381 [M+H]

TLC Rf: 0.33 (EtOAc/cyclohex 1:1)

### EXAMPLE 1.25

### (5-tert-Butyl-isoxazol-3-yl)-[4-(3-chloro-phenylethynyl)-4-hydroxy-piperidin-1-yl]-methanone

MS (LC/MS): 387 [M+H]

TLC Rf: 0.40 (EtOAc/cyclohex 1:1)

### EXAMPLE 1.26

### Benzo[1,3]dioxol-2-yl-[4-(3-chloro-phenylethynyl)-4-hydroxy-piperidin-1-yl]-methanone

MS (LC/MS): 384 [M+H]

TLC Rf: 0.42 (EtOAc/cyclohex 1:1)

### EXAMPLE 1.27

### [4-(3-Chloro-phenylethynyl)-4-hydroxy-piperidin-1-yl]-(3,4-difluoro-phenyl)-methanone

MS (LC/MS): 356 [M+H]

TLC Rf: 0.22 (EtOAc/cyclohex 1:1)

### EXAMPLE 1.28

### [4-(3-Chloro-phenylethynyl)-4-hydroxy-piperidin-1-yl]-oxazol-5-yl-methanone

MS (LC/MS): 331 [M+H]

TLC Rf: 0.22 (EtOAc/cyclohex 1:1)

### EXAMPLE 1.29

### [4-(3-Chloro-phenylethynyl)-4-hydroxy-piperidin-1-yl]-(6-methoxy-pyridin-3-yl)-methanone

MS (LC/MS): 371 [M+H]

TLC Rf: 0.22 (EtOAc/cyclohex 1:1)

### EXAMPLE 1.30

### [4-(3-Chloro-phenylethynyl)-4-hydroxy-piperidin-1-yl]-(2-methoxy-pyridin-3-yl)-methanone

MS (LC/MS): 371 [M+H]

TLC Rf: 0.24 (EtOAc/cyclohex 1:1)

### EXAMPLE 1.31

### [4-(3-Fluoro-phenylethynyl)-4-hydroxy-piperidin-1-yl]-furan-3-yl-methanone

MS (LC/MS): 314 [M+H]

Mp: 67-81° C.

### EXAMPLE 1.32

### [4-(2-Fluoro-phenylethynyl)-4-hydroxy-piperidin-1-yl]-furan-3-yl-methanone

MS (LC/MS): 314 [M+H]

TLC Rf: 0.26 (EtOAc/cyclohex 1:1)

### EXAMPLE 1.33

### [4-(2-Chloro-phenylethynyl)-4-hydroxy-piperidin-1-yl]-furan-3-yl-methanone

MS (LC/MS): 314 [M+H]

TLC Rf: 0.26 (EtOAc/cyclohex 1:1)

### EXAMPLE 1.34

### [4-(4-Chloro-phenylethynyl)-4-hydroxy-piperidin-1-yl]-furan-3-yl-methanone

MS (LC/MS): 330 [M+H]

Mp: 124-134° C.

### EXAMPLE 1.35

### [4-(2,4-Difluoro-phenylethynyl)-4-hydroxy-piperidin-1-yl]-furan-3-yl-methanone

MS (LC/MS): 332 [M+H]

Mp: 80-94° C.

### EXAMPLE 1.36

### Furan-3-yl-[4-hydroxy-4-(3-methoxy-phenylethynyl)-piperidin-1-yl]-methanone

MS (LC/MS): 326 [M+H]

Mp: 83-85° C.

### EXAMPLE 1.37

### [4-(2,5-Dimethyl-phenylethynyl)-4-hydroxy-piperidin-1-yl]-furan-3-yl-methanone

MS (LC/MS): 324 [M+H]

Mp: 110-114° C.

### EXAMPLE 1.38

### [4-(2,3-Difluoro-phenylethynyl)-4-hydroxy-piperidin-1-yl]-furan-3-yl-methanone

MS (LC/MS): 332 [M+H]

TLC Rf: 0.21 (EtOAc/cyclohex 1:1)

### EXAMPLE 1.39

### 3-Fluoro-5-[1-(furan-3-carbonyl)-4-hydroxy-piperidin-4-ylethynyl]-benzonitrile

MS (LC/MS): 339 [M+H]

TLC Rf: 0.28 (EtOAc/cyclohex 2:1)

### EXAMPLE 1.40

### 3-[1-(Furan-3-carbonyl)-4-hydroxy-piperidin-4-ylethynyl]-benzonitrile

MS (LC/MS): 321 [M+H]

TLC Rf: 0.22 (EtOAc/cyclohex 2:1)

### EXAMPLE 1.41

### [4-(3,5-Difluoro-phenylethynyl)-4-hydroxy-piperidin-1-yl]-furan-3-yl-methanone

MS (LC/MS): 332 [M+H]

TLC Rf: 0.34 (EtOAc/cyclohex 1:1)

### EXAMPLE 1.42

### Furan-3-yl-[4-hydroxy-4-(3-trifluoromethyl-phenylethynyl)-piperidin-1-yl]-methanone

MS (LC/MS): 364.5 [M+H]

TLC Rf: 0.45 (cyclohex/EtOAc 4:1)

### EXAMPLE 1.43

### [4-(3,5-Dichloro-phenylethynyl)-4-hydroxy-piperidin-1-yl]-furan-3-yl-methanone

MS (LC/MS): 365.3 [M+H]

TLC Rf: 0.4 (cyclohex/EtOAc 4:1)

### EXAMPLE 1.44

### [4-(3-Difluoromethoxy-phenylethynyl)-4-hydroxy-piperidin-1-yl]-furan-3-yl-methanone

MS (LC/MS): 362.2 [M+H]

TLC Rf: 0.55 (EtOAc)

### EXAMPLE 1.45

### 5-[1-(Furan-3-carbonyl)-4-hydroxy-piperidin-4-ylethynyl]-nicotinonitrile

MS (LC/MS): 322.2 [M+H]

TLC Rf: 0.36 (EtOAc)

### EXAMPLE 1.46

### {4-[3-(3-Chloro-phenyl)-prop-2-ynyl]-4-hydroxy-piperidin-1-yl}-(2,3-dihydro-benzo[1,4]dioxin-2-yl)-methanone

MS (LC/MS): 389 [M+H]

TLC Rf: 0.26 (cyclohex/EtOAc 1:1)

### EXAMPLE 1.47

### [4-(3-Chloro-phenylethynyl)-4-hydroxy-piperidin-1-yl]-((R)-2,2-dimethyl-[1,3]dioxolan-4-yl)-methanone

MS (LC/MS): 364 [M+H]

TLC Rf: 0.19 (cyclohex/EtOAc 1:1)

### EXAMPLE 1.48

### [4-(3-Chloro-phenylethynyl)-4-hydroxy-piperidin-1-yl]-((S)-2,2-dimethyl-[1,3]dioxolan-4-yl)-methanone

MS (LC/MS): 364 [M+H]

TLC Rf: 0.19 (cyclohex/EtOAc 1:1)

### EXAMPLE 1.49

### (5-Chloro-furan-2-yl)-[4-(3-chloro-phenylethynyl)-4-hydroxy-piperidin-1-yl]-methanone

MS (LC/MS): 363 [M+H]

TLC Rf: 0.27 (cyclohex/EtOAc 1:1)

### EXAMPLE 1.50

### [4-(3-Chloro-phenylethynyl)-4-hydroxy-piperidin-1-yl]-(R)-tetrahydro-furan-2-yl-methanone

MS (LC/MS): 334 [M+H]

TLC Rf: 0.09 (cyclohex/EtOAc 1:1)

### EXAMPLE 1.51

### [4-(3-Chloro-phenylethynyl)-4-hydroxy-piperidin-1-yl]-(S)-tetrahydro-furan-2-yl-methanone

MS (LC/MS): 334 [M+H]

TLC Rf: 0.09 (cyclohex/EtOAc 1:1)

### EXAMPLE 1.52

### [4-(3-Chloro-phenylethynyl)-4-hydroxy-piperidin-1-yl]-pyridin-4-yl-methanone

MS (LC/MS): 341 [M+H]

Mp: 171-173° C.

### EXAMPLE 1.53

### [4-(3-Chloro-phenylethynyl)-4-hydroxy-piperidin-1-yl]-(3,5-difluoro-pyridin-2-yl)-methanone

MS (LC/MS): 377 [M+H]

TLC Rf: 0.19 (cyclohex/EtOAc 1:1)

### EXAMPLE 1.54

### [4-(3-Chloro-phenylethynyl)-4-hydroxy-piperidin-1-yl]-(6-methyl-pyridin-2-yl)-methanone

MS (LC/MS): 355.3 [M+H]

TLC Rf: 0.44 (EtOAc)

### EXAMPLE 1.55

### [4-(3-Chloro-phenylethynyl)-4-hydroxy-piperidin-1-yl]-(5-chloro-pyridin-2-yl)-methanone

MS (LC/MS): 375.3 [M+H]

TLC Rf: 0.19 (cyclohex/EtOAc 1:1)

### EXAMPLE 1.56

### [4-(3-Chloro-phenylethynyl)-4-hydroxy-piperidin-1-yl]-(6-chloro-pyridin-2-yl)-methanone

MS (LC/MS): 376.3 [M+H]

TLC Rf: 0.13 (cyclohex/EtOAc 1:1)

### EXAMPLE 1.57

### (5-Chloro-1-methyl-1H-pyrrol-2-yl)-[4-(3-chloro-phenylethynyl)-4-hydroxy-piperidin-1-yl]-methanone

MS (LC/MS): 378.2 [M+H]

TLC Rf: 0.21 (cyclohex/EtOAc 1:1)

### EXAMPLE 1.58

### [4-(3-Chloro-phenylethynyl)-4-hydroxy-piperidin-1-yl]-(5-chloro-1H-pyrrol-2-yl)-methanone

MS (LC/MS): 364.3 [M+H]

TLC Rf: 0.29 (cyclohex/EtOAc 1:1)

### EXAMPLE 1.59

### [4-(3-Chloro-phenylethynyl)-4-hydroxy-piperidin-1-yl]-(1H-methyl-1H-pyrazol-3-yl)-methanone

MS (LC/MS): 344 [M+H]

TLC Rf: 0.22 (EtOAc)

### EXAMPLE 1.60

### [4-(3-Chloro-phenylethynyl)-4-hydroxy-piperidin-1-yl]-(3-fluoro-phenyl)-methanone

A solution of TFFH (tetramethylfluoroformamidinium hexafluorophosphate (24.6 mg, 0.093 mmol) in DMA (0.23 ml) and DIPEA (36 μl, 0.213 mmol) was added to solid 3-fluorobenzoic acid (11.9 mg, 0.085 mmol) under argon atmosphere at room temperature. After stirring for 20 min., a solution of 4-(3-chloro-phenylethynyl)-piperidin-4-ol (21.2 mg, 0.085 mmol) in DMA (0.43 ml) was added and the crude reaction mixture was purified without further treatment after stirring for 24 h on a preparative LC/MS system, yielding the title compound (17.8 mg, 0.050 mmol).

MS (LC/MS): 358 [M+H]

HPLC Rt: 6.78 min (gradient elution)

General LC/MS purification conditions: The crude reaction mixture was injected onto a Waters Atlantis C-18 column (dimensions: 19×100 mm, particle size: 5 μm, pore size: 100 A) and eluted using a 15 ml/min gradient flow rate. The gradient used is as following:

0 min: water containing 0.1% TFA (95%), acetonitrile (5%)
1 min: water containing 0.1% TFA (95%), acetonitrile (5%)
7 min: water containing 0.1% TFA (5%), acetonitrile (95%)
9 min: water containing 0.1% TFA (5%), acetonitrile (95%)

Fractions were triggered by MS detection (ES+mode) of the expected molecular ion peak and UV absorption was measured at 254 nm. The recorded data was processed using the MassLynx 4.0 program from Waters.

Following the same procedure, the following compounds can be obtained:

### EXAMPLE 1.61

### 1-[4-(3-Chloro-phenylethynyl)-4-hydroxy-piperidin-1-yl]-2-(2-methoxy-phenyl)-ethanone

MS (LC/MS): 384 [M+H]

HPLC Rt: 6.84 min (gradient elution)

### EXAMPLE 1.62

### [4-(3-Chloro-phenylethynyl)-4-hydroxy-piperidin-1-yl]-(4-pyrrol-1-yl-phenyl)-methanone

MS (LC/MS): 405 [M+H]

HPLC Rt: 7.15 min (gradient elution)

### EXAMPLE 1.63

### [4-(3-Chloro-phenylethynyl)-4-hydroxy-piperidin-1-yl]-(1-methyl-1H-indol-2-yl)-methanone

MS (LC/MS): 393 [M+H]

HPLC Rt: 7.30 min (gradient elution)

### EXAMPLE 1.64

### [4-(3-Chloro-phenylethynyl)-4-hydroxy-piperidin-1-yl]-(5-hydroxy-1H-indol-2-yl)-methanone

MS (LC/MS): 395 [M+H]

HPLC Rt: 5.70 min (gradient elution)

### EXAMPLE 1.65

### [4-(3-Chloro-phenylethynyl)-4-hydroxy-piperidin-1-yl]-(2,2-dichloro-1-methyl-cyclopropyl)-methanone

MS (LC/MS): 386 [M+H]

HPLC Rt: 7.12 min (gradient elution)

### EXAMPLE 1.66

### 4-[4-(3-Chloro-phenylethynyl)-4-hydroxy-piperidine-1-carbonyl]-benzoic acid methyl ester

MS (LC/MS): 398 [M+H]

HPLC Rt: 6.72 min (gradient elution)

### EXAMPLE 1.67

### [4-(3-Chloro-phenylethynyl)-4-hydroxy-piperidin-1-yl]-(4-hydroxy-3,5-dimethoxy-phenyl)-methanone

MS (LC/MS): 416 [M+H]

HPLC Rt: 6.00 min (gradient elution)

### EXAMPLE 1.68

### 1-[4-(3-Chloro-phenylethynyl)-4-hydroxy-piperidin-1-yl]-2-(2-trifluoromethoxy-phenyl)-ethanone

MS (LC/MS): 438 [M+H]

HPLC Rt: 6.27 min (gradient elution)

### EXAMPLE 1.69

### [4-(3-Chloro-phenylethynyl)-4-hydroxy-piperidin-1-yl]-(4-hydroxy-phenyl)-methanone

MS (LC/MS): 356 [M+H]

HPLC Rt: 5.75 min (gradient elution)

### EXAMPLE 1.70

### [4-(3-Chloro-phenylethynyl)-4-hydroxy-piperidin-1-yl]-(2-hydroxy-phenyl)-methanone

MS (LC/MS): 356 [M+H]

HPLC Rt: 5.89 min (gradient elution)

### EXAMPLE 1.71

### 5-[4-(3-Chloro-phenylethynyl)-4-hydroxy-piperidine-1-carbonyl]-tricyclo[2.2.1.0*2,6*]heptan-3-one

MS (LC/MS): 370 [M+H]

HPLC Rt: 5.71 min (gradient elution)

### EXAMPLE 1.72

### (4-Amino-5-chloro-2-methoxy-phenyl)-[4-(3-chloro-phenylethynyl)-4-hydroxy-piperidin-1-yl]-methanone

MS (LC/MS): 419 [M+H]

HPLC Rt: 6.25 min (gradient elution)

### EXAMPLE 1.73

### (2-Amino-3-chloro-phenyl)-[4-(3-chloro-phenylethynyl)-4-hydroxy-piperidin-1-yl]-methanone

MS (LC/MS): 389 [M+H]

HPLC Rt: 6.69 min (gradient elution)

### EXAMPLE 1.74

### [4-(3-Chloro-phenylethynyl)-4-hydroxy-piperidin-1-yl]-(4-hydroxy-3-methoxy-phenyl)-methanone

MS (LC/MS): 386 [M+H]

HPLC Rt: 5.77 min (gradient elution)

### EXAMPLE 1.75

### [4-(3-Chloro-phenylethynyl)-4-hydroxy-piperidin-1-yl]-(2-fluoro-phenyl)-methanone

MS (LC/MS): 358 [M+H]

HPLC Rt: 6.49 min (gradient elution)

### EXAMPLE 1.76

### [4-(3-Chloro-phenylethynyl)-4-hydroxy-piperidin-1-yl]-(3-dimethylamino-phenyl)-methanone

MS (LC/MS): 383 [M+H]

HPLC Rt: 5.10 min (gradient elution)

### EXAMPLE 1.77

### [4-(3-Chloro-phenylethynyl)-4-hydroxy-piperidin-1-yl]-naphthalen-2-yl-methanone

MS (LC/MS): 390 [M+H]

HPLC Rt: 6.90 min (gradient elution)

### EXAMPLE 1.78

### 1-[4-(3-Chloro-phenylethynyl)-4-hydroxy-piperidin-1-yl]-4-(1H-indol-3-yl)-butan-1-one

MS (LC/MS): 421 [M+H]

HPLC Rt: 6.69 min (gradient elution)

### EXAMPLE 1.79

### 4-[4-(3-Chloro-phenylethynyl)-4-hydroxy-piperidine-1-carbonyl]-benzonitrile

MS (LC/MS): 365 [M+H]

HPLC Rt: 6.25 min (gradient elution)

### EXAMPLE 1.80

### [4-(3-Chloro-phenylethynyl)-4-hydroxy-piperidin-1-yl]-pyridin-2-yl-methanone

MS (LC/MS): 341[M+H]

HPLC Rt: 5.47 min (gradient elution)

### EXAMPLE 1.81

### Adamantan-2-yl-[4-(3-chloro-phenylethynyl)-4-hydroxy-piperidin-1-yl]-methanone

MS (LC/MS): 398 [M+H]

HPLC Rt: 7.86 min (gradient elution)

### EXAMPLE 1.82

### (3-Amino-pyrazin-2-yl)-[4-(3-chloro-phenylethynyl)-4-hydroxy-piperidin-1-yl]-methanone

MS (LC/MS): 357 [M+H]

HPLC Rt: 5.43 min (gradient elution)

### EXAMPLE 1.83

### (6-Amino-pyridin-3-yl)-[4-(3-chloro-phenylethynyl)-4-hydroxy-piperidin-1-yl]-methanone

MS (LC/MS): 356 [M+H]

HPLC Rt: 4.55 min (gradient elution)

### EXAMPLE 1.84

### 4-Amino-N-{4-[4-(3-chloro-phenylethynyl)-4-hydroxy-piperidine-1-carbonyl]-phenyl}-benzamide

MS (LC/MS): 474 [M+H]

HPLC Rt: 5.53 min (gradient elution)

### EXAMPLE 1.85

### N-{4-[4-(3-Chloro-phenylethynyl)-4-hydroxy-piperidine-1-carbonyl]-phenyl}-benzamide

MS (LC/MS): 459 [M+H]

HPLC Rt: 6.43 min (gradient elution)

### EXAMPLE 1.86

### N-{6-[4-(3-Chloro-phenylethynyl)-4-hydroxy-piperidine-1-carbonyl]-benzothiazol-2-yl}-acetamide

MS (LC/MS): 454 [M+H]

HPLC Rt: 5.90 min (gradient elution)

### EXAMPLE 1.87

### [4-(3-Chloro-phenylethynyl)-4-hydroxy-piperidin-1-yl]-(4-fluoro-phenyl)-methanone

MS (LC/MS): 358 [M+H]

HPLC Rt: 6.56 min (gradient elution)

### EXAMPLE 1.88

### (5-Bromo-furan-2-yl)-[4-(3-chloro-phenylethynyl)-4-hydroxy-piperidin-1-yl]-methanone

MS (LC/MS): 408 [M+H]

HPLC Rt: 6.68 min (gradient elution)

### EXAMPLE 1.89

### Benzo[1,3]dioxol-5-yl-[4-(3-chloro-phenylethynyl)-4-hydroxy-piperidin-1-yl]-methanone

MS (LC/MS): 384 [M+H]

HPLC Rt: 6.33 min (gradient elution)

### EXAMPLE 1.90

### 1-[4-(3-Chloro-phenylethynyl)-4-hydroxy-piperidin-1-yl]-2-thiophen-3-yl-ethanone

MS (LC/MS): 360 [M+H]

HPLC Rt: 6.36 min (gradient elution)

### EXAMPLE 1.91

### Acetic acid 4-[4-(3-chloro-phenylethynyl)-4-hydroxy-piperidine-1-carbonyl]-phenyl ester

MS (LC/MS): 398 [M+H]

HPLC Rt: 6.30 min (gradient elution)

### EXAMPLE 1.92

### (3-Chloro-phenyl)-[4-(3-chloro-phenylethynyl)-4-hydroxy-piperidin-1-yl]-methanone

MS (LC/MS): 374 [M+H]

HPLC Rt: 6.80 min (gradient elution)

### EXAMPLE 1.93

### 1-[4-(3-Chloro-phenylethynyl)-4-hydroxy-piperidin-1-yl]-4-phenyl-butane-1,4-dione

MS (LC/MS): 396 [M+H]

HPLC Rt: 6.55 min (gradient elution)

### EXAMPLE 1.94

### [4-(3-Chloro-phenylethynyl)-4-hydroxy-piperidin-1-yl]-pyridin-3-yl-methanone

MS (LC/MS): 341 [M+H]

HPLC Rt: 4.73 min (gradient elution)

### EXAMPLE 1.95

### (5-Bromo-pyridin-3-yl)-[4-(3-chloro-phenylethynyl)-4-hydroxy-piperidin-1-yl]-methanone

MS (LC/MS): 419 [M+H]

HPLC Rt: 6.19 min (gradient elution)

### EXAMPLE 1.96

### (5-Butyl-pyridin-2-yl)-[4-(3-chloro-phenylethynyl)-4-hydroxy-piperidin-1-yl]-methanone

MS (LC/MS): 397 [M+H]

HPLC Rt: 6.58 min (gradient elution)

### EXAMPLE 1.97

### [4-(3-Chloro-phenylethynyl)-4-hydroxy-piperidin-1-yl]-isoquinolin-1-yl-methanone

MS (LC/MS): 391 [M+H]

HPLC Rt: 6.03 min (gradient elution)

### EXAMPLE 1.98

### [4-(3-Chloro-phenylethynyl)-4-hydroxy-piperidin-1-yl]-pyrazin-2-yl-methanone

MS (LC/MS): 342 [M+H]

HPLC Rt: 5.58 min (gradient elution)

### EXAMPLE 1.99

### [4-(3-Chloro-phenylethynyl)-4-hydroxy-piperidin-1-yl]-quinolin-4-yl-methanone

MS (LC/MS): 392 [M+H]

HPLC Rt: 5.78 min (gradient elution)

### EXAMPLE 1.100

### [4-(3-Chloro-phenylethynyl)-4-hydroxy-piperidin-1-yl]-quinolin-2-yl-methanone

MS (LC/MS): 391 [M+H]

HPLC Rt: 6.45 min (gradient elution)

### EXAMPLE 1.101

### [4-(3-Chloro-phenylethynyl)-4-hydroxy-piperidin-1-yl]-(5,6-dichloro-pyridin-3-yl)-methanone

MS (LC/MS): 409[M+H]

HPLC Rt: 6.86 min (gradient elution)

### EXAMPLE 1.102

### [4-(3-Chloro-phenylethynyl)-4-hydroxy-piperidin-1-yl]-(2,6-dimethoxy-pyridin-3-yl)-methanone

MS (LC/MS): 401 [M+H]

HPLC Rt: 6.64 min (gradient elution)

### EXAMPLE 1.103

### [4-(3-Chloro-phenylethynyl)-4-hydroxy-piperidin-1-yl]-cinnolin-4-yl-methanone

MS (LC/MS): 392 [M+H]

HPLC Rt: 5.80 min (gradient elution)

### EXAMPLE 1.104

### [4-(3-Chloro-phenylethynyl)-4-hydroxy-piperidin-1-yl]-quinoxalin-2-yl-methanone

MS (LC/MS): 392 [M+H]

HPLC Rt: 6.39 min (gradient elution)

### EXAMPLE 1.105

### [4-(3-Chloro-phenylethynyl)-4-hydroxy-piperidin-1-yl]-(6-pyrrol-1-yl-pyridin-3-yl)-methanone

MS (LC/MS): 406 [M+H]

HPLC Rt: 6.78 min (gradient elution)

### EXAMPLE 1.106

### [4-(3-Chloro-phenylethynyl)-4-hydroxy-piperidin-1-yl]-[6-(2,2,2-trifluoro-ethoxy)-pyridin-3-yl]-methanone

MS (LC/MS): 439 [M+H]

HPLC Rt: 6.82 min (gradient elution)

### EXAMPLE 1.107

### 6-[4-(3-Chloro-phenylethynyl)-4-hydroxy-piperidine-1-carbonyl]-nicotinic acid methyl ester

MS (LC/MS): 399 [M+H]

HPLC Rt: 6.03 min (gradient elution)

### EXAMPLE 1.8

### [4-(3-Chloro-phenylethynyl)-4-hydroxy-piperidin-1-yl]-(6-chloro-pyridin-3-yl)-methanone

MS (LC/MS): 375[M+H]

HPLC Rt: 6.21 min (gradient elution)

### EXAMPLE 1.109

### (2-Chloro-6-methoxy-pyridin-4-yl)-[4-(3-chloro-phenylethynyl)-4-hydroxy-piperidin-1-yl]-methanone

MS (LC/MS): 405 [M+H]

HPLC Rt: 6.84 min (gradient elution)

### EXAMPLE 1.110

### [4-(3-Chloro-phenylethynyl)-4-hydroxy-piperidin-1-yl]-(1,4,5,6-tetrahydro-cyclopentapyrazol-3-yl)-methanone

MS (LC/MS): 370 [M+H]

HPLC Rt: 5.78 min (gradient elution)

### EXAMPLE 1.111

### 6-[4-(3-Chloro-phenylethynyl)-4-hydroxy-piperidine-1-carbonyl]-pyridine-2-carboxylic acid isopropyl ester

MS (LC/MS): 427 [M+H]

HPLC Rt: 6.47 min (gradient elution)

### EXAMPLE 1.112

### (R)-3-{2-[4-(3-Chloro-phenylethynyl)-4-hydroxy-piperidin-1-yl]-2-oxo-ethyl}-indan-1-one

MS (LC/MS): 408 [M+H]

HPLC Rt: 6.32 min (gradient elution)

### EXAMPLE 1.113

### [4-(3-Chloro-phenylethynyl)-4-hydroxy-piperidin-1-yl]-(1-methyl-1H-indol-3-yl)-methanone

MS (LC/MS): 393 [M+H]

HPLC Rt: 6.65 min (gradient elution)

### EXAMPLE 1.114

### [4-(3-Chloro-phenylethynyl)-4-hydroxy-piperidin-1-yl]-(5-methyl-isoxazol-4-yl)-methanone

MS (LC/MS): 345[M+H]

HPLC Rt: 5.93 min (gradient elution)

### EXAMPLE 1.115

### Benzofuran-3-yl-[4-(3-chloro-phenylethynyl)-4-hydroxy-piperidin-1-yl]-methanone

MS (LC/MS): 380 [M+H]

HPLC Rt: 6.80 min (gradient elution)

### EXAMPLE 1.116

### 4-[4-(3-Chloro-phenylethynyl)-4-hydroxy-piperidine-1-carbonyl]-cyclohexanecarboxylic acid methyl ester

MS (LC/MS): 404 [M+H]

HPLC Rt: 6.30 min (gradient elution)

### EXAMPLE 1.117

### [4-(3-Chloro-phenylethynyl)-4-hydroxy-piperidin-1-yl]-(1H-pyrrol-3-yl)-methanone

MS (LC/MS): 329 [M+H]

HPLC Rt: 5.53 min (gradient elution)

### EXAMPLE 1.118

### 1-[4-(3-Chloro-phenylethynyl)-4-hydroxy-piperidin-1-yl]-2-(2-methoxy-phenoxy)-ethanone

MS (LC/MS): 400 [M+H]

HPLC Rt: 6.45 min (gradient elution)

### EXAMPLE 1.119

### 1-{4-[4-(3-Chloro-phenylethynyl)-4-hydroxy-piperidine-1-carbonyl]-phenyl}-ethanone

MS (LC/MS): 382 [M+H]

HPLC Rt: 6.19 min (gradient elution)

### EXAMPLE 1.120

### [4-(3-Chloro-phenylethynyl)-4-hydroxy-piperidin-1-yl]-(4-methylamino-phenyl)-methanone

MS (LC/MS): 369 [M+H]

HPLC Rt: 5.32 min (gradient elution)

### EXAMPLE 1.121

### [4-(3-Chloro-phenylethynyl)-1-(3,5-dichloro-phenyl)-piperidin-4-ol

To a solution of 3,5-dichlorophenylboronic acid (162 mg, 0.85 mmol, 2 eq), Copper(II) acetate (17.0 mg, 0.085 mmol, 0.2 eq) and moleculare sieves (4 Å, 0.2 g) in DCM (3 ml) under an oxygen atmosphere is added 4-(3-Chloro-phenylethynyl)-piperidin-4-ol (100 mg, 0.42 mmol, 1 eq). After stirring the reaction mixture for 48 h at 40° C. the solution was filtered and the solvent evaporated. Resulting crude material is purified on silica (Flashmaster, EtOAc/hexane) to afford pure product (30 mg, 19%).

MS (LC/MS): 381 [M+H]

TLC Rf: 0.35 (EtOAc/hexane 1:1)

Following the same procedure, the following compounds can be obtained:

### EXAMPLE 1.122

### 1-(3-Chloro-phenyl)-4-(3-chloro-phenylethynyl)-piperidin-4-ol

MS (LC/MS): 347 [M+H]

TLC Rf: 0.33 (EtOAc/hexane 1:4)

### EXAMPLE 1.123

### 1-(4-Chloro-phenyl)-4-(3-chloro-phenylethynyl)-piperidin-4-ol

MS (LC/MS): 347 [M+H]

Mp: 82-86° C.

### EXAMPLE 1.124

### 1-(2-Chloro-phenyl)-4-(3-chloro-phenylethynyl)-piperidin-4-ol

MS (LC/MS): 347 [M+H]

TLC Rf: 0.33 (EtOAc/hexane 1:4)

### EXAMPLE 1.125

### 4-(3-Chloro-phenylethynyl)-1-(4-trifluoromethyl-phenyl)-piperidin-4-ol

MS (LC/MS): 381 [M+H]

Mp: 113-116° C.

### EXAMPLE 1.126

### 3-[4-(3-Chloro-phenylethynyl)-4-hydroxy-piperidin-1-yl]-benzonitrile

MS (LC/MS): 337 [M+H]

TLC Rf: 0.62 (EtOAc/hexane 1:1)

### EXAMPLE 1.127

### 4-(3-Chloro-phenylethynyl)-1-(3-methoxy-phenyl)-piperidin-4-ol

MS (LC/MS): 342 [M+H]

TLC Rf: 0.66 (EtOAc/hexane 1:1)

### EXAMPLE 1.128

### 4-(3-Chloro-phenylethynyl)-1-(4-isopropyl-phenyl)-piperidin-4-ol

MS (LC/MS): 354 [M+H]

Mp: 114-119° C.

### EXAMPLE 1.129

### 4-(3-Chloro-phenylethynyl)-1′-ethyl-[1,3′]bipiperidinyl-4-ol

A solution of 4-(3-Chloro-phenylethynyl)-piperidin-4-ol (70 mg), 1-ethyl-3-piperidone hydrochloride (49 mg), Sodium-triacetoxyborohydride (88.1 mg) and acetic acid (17 μL) in 1,2-dichloroethane (15 ml) was stirred for 18 h at 250. The mixture was distributed between 0.1 M HCl and DCM, the phases separated, the aqueous phase adjusted to pH 10 and extracted with DCM. Organic phases were dried over Na₂SO₄ and evaporated. Chromatography afforded 91 mf of the desired product (88%).

MS (LC/MS): 347 [M+H]

TLC Rf: 0.33 (EtOAc/hexane 1:1)

### EXAMPLE 1.130

### 4-(3-Chloro-phenylethynyl)-1-pyrimidin-2-yl-piperidin-4-ol

A solution of 4-(3-Chloro-phenylethynyl)-piperidin-4-ol (69.7 mg), 2-bromopyrimidine (40 mg), Lithium-bis(trimethylsilyl)amide (540 μM, 1 M in THF), Pd₂(dba)₃ (3.42 mg) and 2-(dicyclohexyl)-biphenylphosphine (2.59 mg) in de-gassed THF (5 ml) was stirred under Ar atmosphere for 18 h at 600. The mixture was distributed between cold 1 M NaHCO₃ and EtOAc, the phases separated, the aqueous phase extracted with EtOAc, the combined organic phases dried over Na₂SO₄ and evaporated. Chromatography afforded 26.8 mf of the desired product (35%).

MS (LC/MS): 314 [M+H]

TLC Rf: 0.31 (EtOAc/hexane 1:1)

Following procedure 1.130, the following compounds can be obtained:

### EXAMPLE 1.131

### 6′-Chloro-4-(3-chloro-phenylethynyl)-3,4,5,6-tetrahydro-2H-[1,2′]bipyridinyl-4-ol

MS (LC/MS): 348 [M+H]

TLC Rf: 0.54 (EtOAc/hexane 1:1)

### EXAMPLE 1.132

### 4-(3-Chloro-phenylethynyl)-1′-oxy-3,4,5,6-tetrahydro-2H-[1,2′]bipyridinyl-4-ol

MS (LC/MS): 329 [M+H]

TLC Rf: 0.24 (DCM/MeOH 9:1)

### EXAMPLE 1.133

### 4′-Bromo-4-(3-chloro-phenylethynyl)-3,4,5,6-tetrahydro-2H-[1,2′]bipyridinyl-4-ol

MS (LC/MS): 392 [M+H]

Mp: 62-65° C.

### EXAMPLE 1.134

### 2′-Bromo-4-(3-chloro-phenylethynyl)-3,4,5,6-tetrahydro-2H-[1,4′]bipyridinyl-4-ol

MS (LC/MS): 392 [M+H]

Mp: 153-156° C.

### EXAMPLE 1.135

### 4-(3-Chloro-phenylethynyl)-1-(3-trifluoromethyl-phenyl)-piperidin-4-ol

MS (LC/MS): 380 [M+H]

TLC Rf: 0.25 (EtOAc/hexane 1:4)

Following procedure 1.129, the following compounds can be obtained:

### EXAMPLE 1.136

### 1-(2-Chloro-benzyl)-4-(3-chloro-phenylethynyl)-piperidin-4-ol

MS (LC/MS): 361 [M+H]

TLC Rf: 0.17 (EtOAc/hexane 1:4)

Following procedure 1.130, the following compounds can be obtained:

### EXAMPLE 1.137

### 4-(3-Chloro-phenylethynyl)-1-o-tolyl-piperidin-4-ol

MS (LC/MS): 326 [M+H]

Mp: 128-130° C.

### EXAMPLE 1.138

### 4-(3-Chloro-phenylethynyl)-3,4,5,6-tetrahydro-2H-[1,3]bipyridinyl-4-ol

MS (LC/MS): 313 [M+H]

Mp: 124-128° C.

### EXAMPLE 1.139

### 4-(3-Chloro-phenylethynyl)-1-quinoxalin-5-yl-piperidin-4-ol

MS (LC/MS): 364 [M+H]

Mp: 68-70° C.

Following procedure 1.129, the following compounds can be obtained:

### EXAMPLE 1.140

### 1-(5-Chloro-3-methyl-1-phenyl-1H-pyrazol-4-ylmethyl)-4-(3-chloro-phenylethynyl)-piperidin-4-ol

MS (LC/MS): 441 [M+H]

TLC Rf: 0.45 (EtOAc/hexane 1:1)

### EXAMPLE 1.141

### 4-(3-Chloro-phenylethynyl)-1-(2,3-dihydro-benzo[1,4]dioxin-6-ylmethyl)-piperidin-4-ol

MS (LC/MS): 384 [M+H]

TLC Rf: 0.51 (EtOAc/hexane 1:1)

Following procedure 1.130, the following compounds can be obtained:

### EXAMPLE 1.142

### 4-(3-Chloro-phenylethynyl)-3,4,5,6-tetrahydro-2H-[1,2′]bipyridinyl-4-ol

MS (LC/MS): 313 [M+H]

TLC Rf: 0.42 (EtOAc/hexane 1:1)

### EXAMPLE 1.143

### 4-(3-Chloro-phenylethynyl)-1-(2,3-dihydro-benzo[1,4]dioxin-6-yl)-piperidin-4-ol

MS (LC/MS): 370 [M+H]

TLC Rf: 0.39 (EtOAc/hexane 2:1)

### EXAMPLE 1.144

### 1-Benzothiazol-2-yl-4-(3-chloro-phenylethynyl)-piperidin-4-ol

MS (LC/MS): 369 [M+H]

Mp: 154-157° C.

### EXAMPLE 1.145

### 4-(3-Chloro-phenylethynyl)-5′-fluoro-3,4,5,6-tetrahydro-2H-[1,2′]bipyridinyl-4-ol

MS (LC/MS): 331 [M+H]

TLC Rf: 0.32 (EtOAc/hexane 1:1)

### EXAMPLE 1.146

### 5′-Chloro-4-(3-chloro-phenylethynyl)-3,4,5,6-tetrahydro-2H-[1,2′]bipyridinyl-4-ol

MS (LC/MS): 349 [M+H]

TLC Rf: 0.44 (EtOAc/hexane 1:1)

### EXAMPLE 1.147

### 4-(3-Chloro-phenylethynyl)-6′-trifluoromethyl-3,4,5,6-tetrahydro-2H-[1,2′]bipyridinyl-4-ol

MS (LC/MS): 381 [M+H]

TLC Rf: 0.47 (EtOAc/hexane 1:1)

### EXAMPLE 1.148

### 4-(3-Chloro-phenylethynyl)-3′-methyl-3,4,5,6-tetrahydro-2H-[1,2′]bipyridinyl-4-ol

MS (LC/MS): 327 [M+H]

Mp: 134-138° C.

### EXAMPLE 1.149

### 4-(3-Chloro-phenylethynyl)-6′-methyl-3,4,5,6-tetrahydro-2H-[1,2′]bipyridinyl-4-ol

MS (LC/MS): 327 [M+H]

TLC Rf: 0.45 (EtOAc/hexane 1:2)

### EXAMPLE 1.150

### 4′-Chloro-4-(3-chloro-phenylethynyl)-3,4,5,6-tetrahydro-2H-[1,2′]bipyridinyl-4-ol

MS (LC/MS): 349 [M+H]

TLC Rf: 0.54 (EtOAc/hexane 1:2)

### EXAMPLE 1.151

### 2′-Chloro-4-(3-chloro-phenylethynyl)-3,4,5,6-tetrahydro-2H-[1,4′]bipyridinyl-4-ol

MS (LC/MS): 349 [M+H]

Mp: 139-142° C.

### EXAMPLE 1.152

### 4-(3-Chloro-phenylethynyl)-4′-trifluoromethyl-3,4,5,6-tetrahydro-2H-[1,2′]bipyridinyl-4-ol

MS (LC/MS): 381 [M+H]

TLC Rf: 0.40 (EtOAc/hexane 1:2)

### EXAMPLE 1.153

### 4-(3-Chloro-phenylethynyl)-1-(6-chloro-pyrimidin-4-yl)-piperidin-4-ol

MS (LC/MS): 349 [M+H]

TLC Rf: 0.38 (EtOAc/hexane 1:2)

### EXAMPLE 1.154

### 1-[4-(3-Chloro-phenylethynyl)-4-hydroxy-3,4,5,6-tetrahydro-2H-[1,2′]bipyridinyl-6′-yl]-ethanone

MS (LC/MS): 356 [M+H]

TLC Rf: 0.36 (EtOAc/hexane 1:2)

### EXAMPLE 1.155

### 4-(3-Chloro-phenylethynyl)-5′-trifluoromethyl-3,4,5,6-tetrahydro-2H-[1,2′]bipyridinyl-4-ol

MS (LC/MS): 381 [M+H]

TLC Rf: 0.45 (EtOAc/hexane 1:2)

### EXAMPLE 1.156

### 5′-Chloro-4-(3-chloro-phenylethynyl)-3,4,5,6-tetrahydro-2H-[1,3′]bipyridinyl-4-ol

MS (LC/MS): 348 [M+H]

Mp: 134-136° C.

### EXAMPLE 1.157

### 4-(3-Chloro-phenylethynyl)-1-(6-chloro-pyrazin-2-yl)-piperidin-4-ol

MS (LC/MS): 349 [M+H]

TLC Rf: 0.36 (EtOAc/hexane 1:2)

### EXAMPLE 1.158

### 4′,6′-Dichloro-4-(3-chloro-phenylethynyl)-3,4,5,6-tetrahydro-2H-[1,2′]-bipyridinyl-4-ol

MS (LC/MS): 382 [M+H]

TLC Rf: 0.33 (EtOAc/hexane 1:2)

### EXAMPLE 1.159

### 2′,6′-Dichloro-4-(3-chloro-phenylethynyl)-3,4,5,6-tetrahydro-2H-[1,4′]-bipyridinyl-4-ol

MS (LC/MS): 382 [M+H]

TLC Rf: 0.25 (EtOAc/hexane 1:2)

### EXAMPLE 1.160

### 3′-Chloro-4-(3-chloro-phenylethynyl)-3,4,5,6-tetrahydro-2H-[1,4′]bipyridinyl-4-ol

MS (LC/MS): 348 [M+H]

TLC Rf: 0.30 (EtOAc/hexane 1:2)

### EXAMPLE 1.161

### 4-(3-Chloro-phenylethynyl)-6′-methyl-4′-trifluoromethyl-3,4,5,6-tetrahydro-2H-[1,2′]bipyridinyl-4-ol

MS (LC/MS): 395 [M+H]

TLC Rf: 0.50 (EtOAc/hexane 1:2)

### EXAMPLE 1.162

### 4-(3-Chloro-phenylethynyl)-1-pyrimidin-5-yl-piperidin-4-ol

MS (LC/MS): 314 [M+H]

Mp: 173-177° C.

### EXAMPLE 1.163

### 4-(3-Chloro-phenylethynyl)-1-imidazo[1,2-a]pyridin-5-yl-piperidin-4-ol

MS (LC/MS): 352 [M+H]

TLC Rf: 0.50 (DCM/MeOH 85:15)

### EXAMPLE 1.164

### 4-(3-Chloro-phenylethynyl)-2′-methyl-3,4,5,6-tetrahydro-2H-[1,3′]-bipyridinyl-4-ol

MS (LC/MS): 327 [M+H]

Mp: 98-102° C.

### EXAMPLE 1.165

### 4 4-(3-Chloro-phenylethynyl)-5′-methyl-3,4,5,6-tetrahydro-2H-[1,3′]-bipyridinyl-4-ol

MS (LC/MS): 327 [M+H]

Mp: 145-150° C.

### EXAMPLE 1.166

### 4 4-(3-Chloro-phenylethynyl)-4′-methyl-3,4,5,6-tetrahydro-2H-[1,2′]-bipyridinyl-4-ol

MS (LC/MS): 327 [M+H]

TLC Rf: 0.47 (EtOAc/hexane 1:1)

### EXAMPLE 2

### (4-tert-Butyl-phenyl)-[3-(3-chloro-phenylethynyl)-3-hydroxy-piperidin-1-yl]-methanone

A solution of 3-(3-Chloro-phenylethynyl)-piperidin-3-ol (59 mg, 0.25 mmol) and 4-tert-butyl-benzoic acid (44.5 mg, 0.25 mmol) in DMF (2 ml) was treated with Et₃N (175 ul ml, 1.25 mmol) and HOBt (37.5 mg, 0.275 mmol), and EDC (54 mg, 0.275 mmol) was added. After shaking for 24 h, the reaction was dissolved in water and extract for three times with tert.butylmethylether. The combined organic layers were washed with 1 M hydrochloric acid (1×10 ml), saturated NaHCO₃ (1×10 ml) and brine (1×10 ml) and dried over Na₂SO₄. Filtration and evaporation of the solvents afforded a yellowish foam (99 mg). Chromatography on a preparative LC-MS (column Waters SunFire C18, 19×100 mm, 5 um; fractionated by mass) with water (+0.1% AcOH)/acetonitrile (+0.1% AcOH) gradient (0-100% acetonitrile in 10 min.) and evaporation of fractions afforded a white foam (49 mg, 50%).

MS (LC/MS): 395.9 [M+]

TLC Rf: 0.28 (Cyclohexane/EtOAc 60/40).

The starting material was prepared as described hereafter:

### 3-(3-Chloro-phenylethynyl)-piperidin-3-ol

A solution of 1-chloro-3-ethynyl-benzene (7.07 g, 50.2 mmol) in THF (120 ml) was cooled to −75° C. Within 30 minutes, a solution of n-BuLi in hexane (1.5 N, 58 ml, 87 mmol) was added and the mixture stirred for 30 minutes at −75° C. A solution of 3-oxo-piperidine-1-carboxylic acid tert-butyl ester (10.0 g, 50.2 mmol) in THF (60 ml) was added dropwise within 45 minutes at −75° C. The cooling bath was removed, and when the mixture had reached room temperature it was slowly poured into a stirred mixture of ice water (1000 ml) and ethylacetate (500 ml). The aqueous phase was separated and extracted twice with ethylacetate (250 ml). The combined organic phases were washed with water (250 ml), dried over Na₂SO₄, filtered and the solvent evaporated to afford 3-(3-chloro-phenylethynyl)-3-hydroxy-piperidine-1-carboxylic acid tert-butyl ester as a yellowish oil crystallization from cyclohexane yielded white crystals.

M.p. 127.7-129.4° C.

This Boc-protected amine (12.50 g, 37.2 mmol) was dissolved in EtOAc (125 ml) and cooled to 0° C. A 2 N solution of HCl in diethylether (230 ml, 470 mmol) was added in one portion. After stirring this mixture for a total of 18 h at room temperature, the white precipitate was filtered off and washed with diethylether. The white solid was poured into a 2N ammoniumhydroxyd solution and extract three times with ethylacetate (3×250 ml). The combined organic phases were dried over Na₂SO₄, filtered and the solvent evaporated to a small volume. Cyclohexane was added and the precipitate was filtered off and dried at high vacuum to give 3-(3-chloro-phenylethynyl)-piperidin-3-ol (8.51 g, 97%) as white crystals.

M.p. 113.3-113.8° C.

Following the same procedure, the following compounds can be obtained:

### EXAMPLE 2.1

### Benzofuran-2-yl-[3-(3-chloro-phenylethynyl)-3-hydroxy-piperidin-1-yl]-methanone

MS (LC/MS): 379.9 [M+]

TLC Rf: 0.26 (Cyclohexane/EtOAc 60/40)

### EXAMPLE 2.2

### [3-(3-Chloro-phenylethynyl)-3-hydroxy-piperidin-1-yl]-pyridin-4-yl-methanone

MS (LC/MS): 340.9 [M+]

TLC Rf: 0.07 (Cyclohexane/EtOAc 20/80)

### EXAMPLE 2.3

### [3-(3-Chloro-phenylethynyl)-3-hydroxy-piperidin-1-yl]-(2,6-dichloro-phenyl)-methanone

MS (LC/MS): 409.8 [M+H]

TLC Rf: 0.34 (Cyclohexane/EtOAc 60/40)

### EXAMPLE 2.4

### (4-Amino-5-chloro-2-methoxy-phenyl)-[3-(3-chloro-phenylethynyl)-3-hydroxy-piperidin-1-yl]-methanone

MS (LC/MS): 419 [M+]

TLC Rf: 0.21 (Cyclohexane/EtOAc 60/40)

### EXAMPLE 2.5

### [3-(3-Chloro-phenylethynyl)-3-hydroxy-piperidin-1-yl]-pyridin-3-yl-methanone

MS (LC/MS): 341 [M+H]

TLC Rf: 0.07 (Cyclohexane/EtOAc 20/80)

### EXAMPLE 2.6

### [3-(3-Chloro-phenylethynyl)-3-hydroxy-piperidin-1-yl]-(2,4-dichloro-phenyl)-methanone

MS (LC/MS): 410 [M+H]

TLC Rf: 0.27 (Cyclohexane/EtOAc 60/40)

### EXAMPLE 2.7

### [3-(3-Chloro-phenylethynyl)-3-hydroxy-piperidin-1-yl]-(2,4-dimethoxy-phenyl)-methanone

MS (LC/MS): 399.9 [M+]

TLC Rf: 0.27 (Cyclohexane/EtOAc 20/80)

### EXAMPLE 2.8

### [3-(3-Chloro-phenylethynyl)-3-hydroxy-piperidin-1-yl]-(2,3-dimethoxy-phenyl)-methanone

MS (LC/MS): 399.9 [M+]

TLC Rf: 0.12 (Cyclohexane/EtOAc 60/40)

### EXAMPLE 2.9

### [3-(3-Chloro-phenylethynyl)-3-hydroxy-piperidin-1-yl]-(2-hydroxy-5-methyl-phenyl)-methanone

MS (LC/MS): 369.9 [M+]

TLC Rf: 0.18 (Cyclohexane/EtOAc 60/40)

### EXAMPLE 2.10

### [3-(3-Chloro-phenylethynyl)-3-hydroxy-piperidin-1-yl]-(3,4-dimethyl-phenyl)-methanone

MS (LC/MS): 367.9 [M+]

TLC Rf: 0.21 (Cyclohexane/EtOAc 60/40)

### EXAMPLE 2.11

### Acetic acid 4-[3-(3-chloro-phenylethynyl)-3-hydroxy-piperidine-1-carbonyl]-phenyl ester

MS (LC/MS): 398 [M+H]

TLC Rf: 0.35 (Cyclohexane/EtOAc 20/80)

### EXAMPLE 2.12

### (4-Chloro-phenyl)-[3-(3-chloro-phenylethynyl)-3-hydroxy-piperidin-1-yl]-methanone

MS (LC/MS): 374 [M+]

TLC Rf: 0.21 (Cyclohexane/EtOAc 60/40)

### EXAMPLE 2.13

### [3-(3-Chloro-phenylethynyl)-3-hydroxy-piperidin-1-yl]-(4-iodo-phenyl)-methanone

MS (LC/MS): 465.8 [M+]

TLC Rf: 0.22 (Cyclohexane/EtOAc 60/40)

### EXAMPLE 2.14

### 3-(3-Chloro-phenylethynyl)-3-hydroxy-piperidine-1-carboxylic acid 1-(4-bromo-phenyl)-ethyl ester

To a solution of 4-bromo-alpha-methylbenzyl alcohol (111 mg, 0.50 mmol) and Et₃N (105 ul ml, 0.55 mmol) in dichloromethane (5 ml) was added di-(N-succinimidyl)carbonate (169 mg, 0.75 mmol. The suspension was stirred for two hours at room temperature and then 3-(3-chloro-phenylethynyl)-piperidin-3-ol (118 mg, 0.50 mmol) and Et₃N (210 ul ml, 1.50 mmol) was added. The reaction was stirred for another two hours and the clear solution was directly purified over a Flash column with cyclohexane/ethylacetate to afford a colorless resin (58 mg, 25%).

MS (LC/MS): 485.8 [M+Na]

TLC Rf: 0.33 (Cyclohexane/EtOAc 60/40)

Following procedure 2.14, the following compounds can be obtained:

### EXAMPLE 2.15

### 3-(3-Chloro-phenylethynyl)-3-hydroxy-piperidine-1-carboxylic acid 1-(4-trifluoromethyl-phenyl)-ethyl ester

MS (LC/MS): 474 [M−H+Na]

TLC Rf: 0.35 (Cyclohexane/EtOAc 60/40)

### EXAMPLE 2.16

### 3-(3-Chloro-phenylethynyl)-3-hydroxy-piperidine-1-carboxylic acid 1-(2-chloro-phenyl)-ethyl ester

MS (LC/MS): 440 [M−H—H+ Na]

TLC Rf: 0.37 (Cyclohexane/EtOAc 60/40)

### EXAMPLE 2.17

### 3-(3-Chloro-phenylethynyl)-3-hydroxy-piperidine-1-carboxylic acid 1-(4-methoxy-phenyl)-ethyl ester

MS (LC/MS): 436 [M−H+Na]

TLC Rf: 0.30 (Cyclohexane/EtOAc 60/40)

### EXAMPLE 2.18

### 3-(3-Chloro-phenylethynyl)-3-hydroxy-piperidine-1-carboxylic acid 1-o-tolyl-ethyl ester

MS (LC/MS): 420 [M−H+Na]

TLC Rf: 0.38 (Cyclohexane/EtOAc 60/40)

### EXAMPLE 2.19

### 3-(3-Chloro-phenylethynyl)-3-hydroxy-piperidine-1-carboxylic acid 1-(4-chloro-phenyl)-ethyl ester

MS (LC/MS): 440 [M−H+Na]

TLC Rf: 0.35 (Cyclohexane/EtOAc 60/40)

### EXAMPLE 2.20

### 3-(3-Chloro-phenylethynyl)-3-hydroxy-piperidine-1-carboxylic acid 1-p-tolyl-ethyl ester

MS (LC/MS): 420 [M−H+Na]

TLC Rf: 0.37 (Cyclohexane/EtOAc 60/40)

### EXAMPLE 2.21

### 5-[3-(3-Chloro-phenylethynyl)-3-hydroxy-piperidin-1-yl]-2-nitro-4-trifluoromethyl-benzoic acid methyl ester

A solution of 3-(3-chloro-phenylethynyl)-piperidin-3-ol (118 mg, 0.50 mmol) and 5-fluoro-2-nitro-4-trifluoromethyl-benzoic acid methyl ester (134 mg, 0.50 mmol) in ethanol (2 ml) was stirred in a microwave oven at 170° C. for 30 minutes. After cooling the reaction, the solvent was evaporated. Resulting crude product is purified on silica (Flashmaster, EtOAc/cyclohexane) to afford pure product (50 mg, 20%).

MS (LC/MS): 505 [M+Na]

TLC Rf: 0.30 (Cyclohexane/EtOAc 60/40).

### EXAMPLE 3

### [3-(3-Chloro-phenylethynyl)-3-hydroxy-pyrrolidin-1-yl]-furan-2-yl-methanone

To a solution of 3-(3-Chloro-phenylethynyl)-3-hydroxy-pyrrolidine-1-carboxylic acid tert-butyl ester (150 mg, 0.68 mmol) and Furane-2-carboxylic acid (91.0 mg, 0.81 mmol, 1.2 eq) in DMF (10 ml) is added sequentially EDC (143 mg, 0.75 mmol, 1.1 eq), HOBt (103 mg, 0.75 mmol, 1.1 eq) and triethylamine (0.47 ml, 3.38 mmol, 5 eq). After stirring the reaction mixture at rt for 23 h, 1 N aqueous HCl (5 ml) is added and the solution is extracted with EtOAc (3×10 ml). Combined organic layers are washed with 10% aqueous hydrogen carbonate solution (5 ml) dried over sodium sulfate and the solvent is evaporated. Resulting crude product is purified on silica (Flashmaster, EtOAc/hexane) to afford pure amide (60 mg, 28%).

MS (LC/MS): 316 [M+H]

TLC Rf: 0.47 (EtOAc)

The starting materials can be prepared as described hereafter:

### i) 3-(3-Chloro-phenylethynyl)-3-hydroxy-pyrrolidine-1-carboxylic acid tert-butyl ester

To a solution of n-butyl lithium (11.6 ml of 1.6 M solution in hexane, 19 mmol, 1.01 eq) in THF (190 ml) at −70° C. under argon is added a solution of 1-Chloro-3-ethynyl-benzene (3.2 ml, 19.2 mmol, 1.05 eq) in THF (30 ml). After stirring the reaction mixture for 30 minutes at −70° C. a solution of 3-oxo-pyrrolidine-1-carboxylic acid tert-butyl ester (2.50 g, 18.3 mmol, 1 eq) in THF (30 ml) was added and the mixture is stirred for another 2.5 h. The solution is diluted with 10% aqueous ammonium chloride solution (10 ml) and EtOAc (50 ml). The organic layer is washed with 1 N aqueous HCl solution (3×50 ml) dried over sodium sulfate and the solvent is evaporated. Resulting crude product is purified on silica (Flashmaster, EtOAc/hexane) to afford pure product (3.38 g, 57%).

### MS (LC/MS): 344 [M+Na]

Mp: 94-104° C.

### ii) 3-(3-Chloro-phenylethynyl)-pyrrolidin-3-ol

3-(3-Chloro-phenylethynyl)-3-hydroxy-pyrrolidine-1-carboxylic acid tert-butyl ester (3.3 g, 10.3 mmol) is dissolved in 4M HCl in dioxane (10 ml) and stirred at rt for 6 h. The solvent is evaporated to afford crude product (2.31 g, 100%) which was directly used without further purification.

MS (LC/MS): 222 [M+H]

Mp: 153-160° C.

Following the same synthetic procedure the following examples can be made:

### EXAMPLE 3.1

### [3-(3-Chloro-phenylethynyl)-3-hydroxy-pyrrolidin-1-yl]-pyridin-2-yl-methanone

MS (LC/MS): 328 [M+H]

TLC Rf: 0.34 (EtOAc)

### EXAMPLE 3.2

### [3-(3-Chloro-phenylethynyl)-3-hydroxy-pyrrolidin-1-yl]-furan-3-yl-methanone

MS (LC/MS): 316 [M+H]

TLC Rf: 0.49 (EtOAc)

### EXAMPLE 3.3

### [3-(3-Chloro-phenylethynyl)-3-hydroxy-pyrrolidin-1-yl]-quinoxalin-2-yl-methanone

MS (LC/MS): 378 [M+H]

TLC Rf: 0.37 (DCM/methanol=95/5)

### EXAMPLE 3.4

### [3-(3-Chloro-phenylethynyl)-3-hydroxy-pyrrolidin-1-yl]-pyridin-2-yl-methanone

MS (LC/MS): 327 [M+H]

TLC Rf: 0.33 (EtOAc)

### EXAMPLE 3.5

### [3-(3-Chloro-phenylethynyl)-3-hydroxy-pyrrolidin-1-yl]-pyridin-3-yl-methanone

MS (LC/MS): 327 [M+H]

TLC Rf: 0.12 (EtOAc)

### EXAMPLE 3.6

### [3-(3-Chloro-phenylethynyl)-3-hydroxy-pyrrolidin-1-yl]-pyridin-4-yl-methanone

MS (LC/MS): 327 [M+H]

TLC Rf: 0.12 (EtOAc)

### EXAMPLE 3.7

### Benzofuran-2-yl-[3-(3-chloro-phenylethynyl)-3-hydroxy-pyrrolidin-1-yl]-methanone

MS (LC/MS): 366 [M+H]

TLC Rf: 0.20 (EtOAc)

### EXAMPLE 3.8

### 3-(3-Chloro-phenylethynyl)-3-hydroxy-pyrrolidin-1-yl]-(5-methyl-pyrazin-2-yl)-methanone

MS (LC/MS): 342 [M+H]

TLC Rf: 0.22 (EtOAc)

### EXAMPLE 3.9

### [3-(3-Chloro-phenylethynyl)-3-hydroxy-pyrrolidin-1-yl]-(tetrahydro-furan-3-yl)-methanone

MS (LC/MS): 320 [M+H]

TLC Rf: 0.16 (EtOAc)

### EXAMPLE 3.10

### [3-(3-Chloro-phenylethynyl)-3-hydroxy-pyrrolidin-1-yl]-(tetrahydro-furan-2-yl)-methanone

MS (LC/MS): 320 [M+H]

TLC Rf: 0.21 (EtOAc)

### EXAMPLE 3.11

### Benzo[1,3]dioxol-2-yl-[3-(3-chloro-phenylethynyl)-3-hydroxy-pyrrolidin-1-yl]-methanone

MS (LC/MS): 370 [M+H]

Mp: 153-155° C.

We claim:

1. A compound of formula (I) 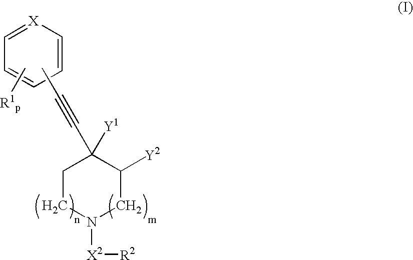
wherein
m represents 0 and n represents 1 or
m represents 0 and n represents 2 or
m represents 1 and n represents 1;
p represents 0, 1, 2, 3, 4 or 5;
X represents CH, N;
X² represents a single bond or an alkandiyl-group, optionally interrupted by one or more oxygen atoms or carbonyl groups or carbonyloxy groups
Y¹ represents OH and Y² represents H or
Y¹ and Y² form a bond;
R¹ represents halogen, cyano, nitro, —CHO, alkyl, alkoxy, halogenalkoxy, halogenalkyl, —C(O)R⁴, —COOR⁴ wherein R⁴ is alkyl or two substituents R¹ together form a alkandiyl or alkenediyl-moiety;
R² represents an unsubstituted or substituted heterocycle, or
R² represents phenyl or substituted phenyl, or
R² represents C(O)R³ wherein R³ represents alkyl, alkoxy or substituted alkoxy, phenyl or substituted phenyl, an unsubstituted or substituted aliphatic heterocycle, an unsubstituted or substituted partly saturated heterocycle containing less than 12 ring atoms, an unsubstituted or substituted aromatic heterocycle containing less than 12 ring atoms or
R² represents C(O)R³ wherein R³ represents unsubstituted or substituted cycloalkyl
R² represents CH₂R⁶, SR⁶, S(O)R⁶, S(O)₂R⁶ wherein R⁶ represents an unsubstituted or substituted heterocycle
in free base or acid addition salt form.

2. The compound of formula (I) according to claim
wherein
m represents 0 and n represents 1 or
m represents 0 and n represents 2 or
m represents 1 and n represents 1;
p represents 0, 1, 2, 3, 4 or 5;
X represents CH, N;
X² represents a single bond;
Y¹ represents OH and Y² represents H or
Y¹ and Y² form a bond;
R¹ represents halogen, cyano, nitro, —CHO, alkyl, alkoxy, halogenalkyl, —C(O)R⁴, —COOR⁴ wherein R⁴ is alkyl or two substituents R¹ together form a alkandiyl or alkenediyl-moiety;
R² represents an unsubstituted or substituted heterocycle, or
R² represents phenyl or substituted phenyl, or
R² represents C(O)R³ wherein R³ represents alkyl, alkoxy or substituted alkoxy, phenyl or substituted phenyl, an unsubstituted or substituted aliphatic heterocycle, an unsubstituted or substituted partly saturated heterocycle containing less than 12 ring atoms, an unsubstituted or substituted aromatic heterocycle containing less than 12 ring atoms or
R² represents CH₂R⁶, SR⁶, S(O)R⁶, S(O)₂R⁶ wherein R⁶ represents an unsubstituted or substituted heterocycle
in free base or acid addition salt form.

3. The compound according to claim 2 represented by formula (I-I) 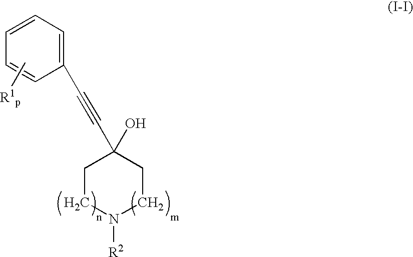
wherein m, n, p, R¹ and R² are as defined in claim 2 in free base or acid addition salt form.

4. A compound of formula (I) according to claim 1 wherein R¹ represents chloro and p represents 1.

5. The trans-Isomer of a compound of formula (I) according to claim 1.

6. A process for the preparation of a compound of formula (I) as defined in claim 1, or a salt thereof, which comprises
i) reacting a compound of formula (II) 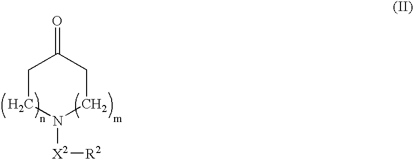
wherein X², R², m, n are as defined above, with a compound of formula (III) 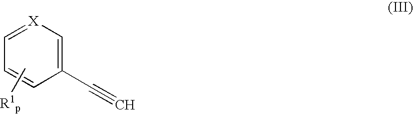
wherein R¹, X and p are as defined above, in the presence of a base, resulting in compounds of formula (I) wherein Y¹ represents OH and Y² represents H; or
ii) - in case X² represents a single bond - reacting a compound of formula (IV) 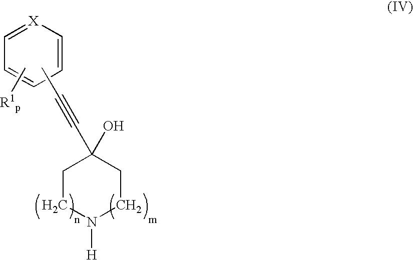
wherein R¹, X, m, n and p are as defined above, with a compound of formula (V)
LG-R² (V)
wherein R² is as defined above and LG represents a leaving group, e.g. a halogen such as Br or Cl, optionally in the presence of reaction auxiliaries, optionally in the presence of a diluent; or
iii) - in case X² represents a single bond - reacting a compound of formula (IV) 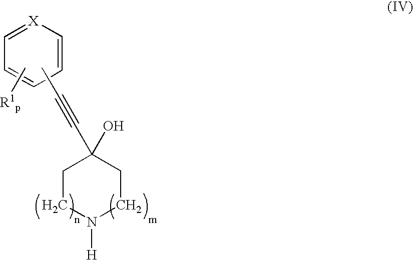
wherein R¹, X, m, n and p are as defined above, with a compound of formula (VI) 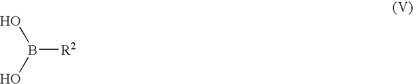
wherein R² is as defined above, optionally in the presence of reaction auxiliaries, optionally in the presence of a diluent; or
iv) reacting a compound of formula (IV) wherein R¹, X, m, n and p are as defined above by reductive amination with a compound of formula (VII) 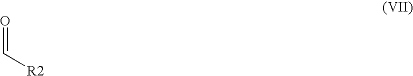
wherein R² is defined as above, or
v) - in case represents carbonyl - reacting a compound of formula (IV) 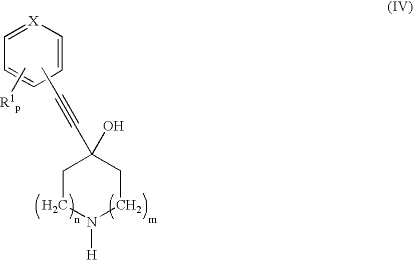
wherein R¹, X, m, n and p are as defined above, with a compound of formula (IIX) 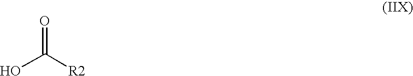
wherein R² is defined as above, optionally in the presence of reaction auxiliaries, optionally in the presence of a diluent and
vi) optionally converting the substituent X²—R² into another substituent X²—R² according to conventional procedures; and
vii) optionally eliminating H₂O from the thus obtained compound resulting in a compound of formula (I) wherein Y¹ and Y² form a bond and
viii) recovering the resulting compound of formula (I) in free base or acid addition salt form.

7. A compound of formula (IV) 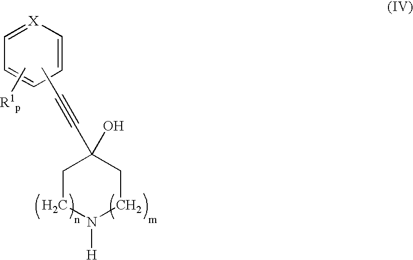
wherein
m represents 0 and n represents 1 or
m represents 0 and n represents 2 or
m represents 1 and n represents 1;
p represents 0, 1, 2, 3, 4 or 5;
X represents CH, N;
R¹ represents halogen, cyano, nitro, —CHO, alkyl, alkoxy, halogenalkoxy, halogenalkyl, —C(O)R⁴, —COOR⁴ wherein R⁴ is alkyl or two substituents R¹ together form a alkandiyl or alkenediyl-moiety;
in free base or acid addition salt form.

8. A process for the preparation of a compound of formula (IV) as defined in claim 4, or a salt thereof which comprises reacting a compound of formula (III) 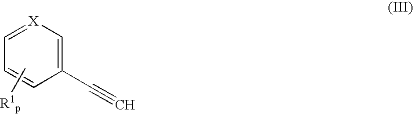
Wherein R¹ and X are as defined in claim 1 with a compound of formula (VI) 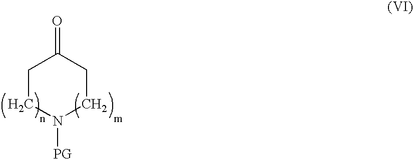
wherein m and n are as define above and PG represents a protecting group, in the presence of a base, optionally in the presence of a diluent.

9. (canceled)

10. A method of antagonizing a mGluR5 receptor in a patient in need thereof, comprising:
administering a therapeutically effective amount of the compound of claim 1 in free base or pharmaceutically acceptable acid addition salt form.

11. A pharmaceutical composition, comprising:
a compound of claim 1 in free base or pharmaceutically acceptable acid addition salt form, and
a pharmaceutical carrier or diluent.

12-13. (canceled)

14. A method of treating disorders associated with irregularities of the glutamatergic signal transmission, and nervous system disorders mediated full or in part by mGluR5, comprising:
administering to a subject in need of such treatment a therapeutically effective amount of a compound of claim 1 in free base or pharmaceutically acceptable acid addition salt form.
